(19) United States
(12) Patent Application Publication
(10) Pub. No.: US 2018/0036424 A1
(43) Pub. Date: Feb. 8, 2018

(54) IMMUNE SYSTEM ENHANCING IMMUNOTHERAPY FOR THE TREATMENT OF CANCER

(71) Applicant: Panacea Pharmaceuticals, Gaithersburg, MD (US)

(72) Inventor: HOSSEIN GHANBARI, Potomac, MD (US)

(21) Appl. No.: 15/590,472

(22) Filed: May 9, 2017

Related U.S. Application Data
(63) Continuation of application No. 13/908,224, filed on Jun. 3, 2013, now abandoned.

(51) Int. Cl. A61K 47/68; A61K 39/295

(52) U.S. Cl. CPC A61K 47/6811; A61K 47/6807

(57) ABSTRACT

This application discloses immunoconjugates comprising antibodies against a particular target (such as cancer associated antigen or cancer specific antigen) that are conjugated with an immune enhancer, recruiter or solicitor. Also discloses are compositions and methods of using the inventive immunoconjugates to treat cancer.

### FIELD OF THE INVENTION

Embodiments of the present disclosure are related to antibodies against a particular target (such as cancer associated antigen or cancer specific antigen) that are conjugated with an immune enhancer, recruiter or solicitor and compositions and methods thereof.

### BACKGROUND OF THE DISCLOSURE

The treatment of cancer has progressed significantly with the development of pharmaceuticals that more efficiently target and kill cancer cells. To this end, researchers have taken advantage of cell-surface receptors and antigens selectively expressed by cancer cells to develop drugs based on antibodies that bind the tumor-specific or tumor-associated antigens. Cytotoxic molecules such as bacteria and plant toxins, radionuclides, and certain chemotherapeutic drugs have been chemically linked to monoclonal antibodies that bind tumor-specific or tumor-associated cell surface antigens. Such compounds are typically referred to as toxin, radionuclide, and drug “conjugates,” respectively. Often they also are referred to as immunoconjugates, radioimmunoconjugates and immunotoxins.

Despite the tumor selectivity afforded by drug conjugates, the use of toxins, radionuclides, and chemotherapeutic drugs continues to present several disadvantages in the clinical context. First, the manufacture of the drug conjugates are often hindered by stability issues such that the drug conjugate composition may be less stable than compositions containing the tumor-specific antibody alone. Second, the size of the drug conjugates may interfere with the binding affinity/specificity of the antibody component.

Thus, there remains a need for development of pharmaceuticals that more efficiently target and kill cancer cells and that seek to minimize the issues that exist with currently available drug conjugate compositions. There also remains a need for methods of using such specifically targeted pharmaceuticals to treat human diseases associated with cell proliferation, such as cancer.

### SUMMARY OF THE INVENTION

Disclosed herein is a novel immune system enhancing immunotherapy involving an antibody against a tumor-associated antigen (TAA) or tumor-specific antigen (TSA) that is conjugated to an immune enhancer analogous to potent toxins. In this system, target cells are eliminated by natural cytotoxic processes of the immune system rather than being killed by toxins. The immune enhancer may be one of an antigenic protein, glycoprotein or polysaccharide derived from a virus, bacteria, or other microorganism.

Embodiments of the present invention contemplate immunoconjugates comprising an antibody and one or more immune enhancers, wherein the antibody is specific for a tumor antigen, and wherein the immune enhancer is an antigen derived from a viral entity or bacteria.

The present invention further contemplates immunoconjugates comprising one or more immune enhancers derived from a viral entity or bacteria, wherein the viral entity is a non-infectious, non-replicating virus, viral particle, virus-like particle (VLP), or antigenic component thereof.

According to some embodiments, an antibody to the tumor antigen aspartyl (asparaginyl) beta-hydroxylase (HAAH) polypeptide is conjugated with one or more immune enhancers. The immune enhancer may be a non-infectious, non-replicating virus or viral particle, for example, a bacteriophage or bacteriophage particle (e.g., lambda phage or lambda phage particle). In some embodiments, the immune enhancer may be a mycobacterial antigen such a tuberculosis (TB) antigen.

According to some embodiments, an antibody to the tumor antigen aspartyl (asparaginyl) beta-hydroxylase (HAAH) polypeptide is conjugated to both a non-replicating Lambda virus and TB antigens, as follows:

(Anti-HAAH)-(Non-Replicating Lambda Virus)-(TB Antigens).

This entity will bind to cancer cells expressing HAAH on their surface and subsequently recruit immune system components resulting in the cancer cell elimination without the negative effects associated with toxin.

The antibody and/or TB antigen may be chemically conjugated to the virus (e.g., Lambda virus) or, alternatively, the virus may be engineered to express the antibody (e.g., single chain fragment) and/or TB antigen on its surface.

### DETAILED DESCRIPTION OF THE INVENTION

This application discloses immune system enhancing immunotherapy compositions and methods related to immunoconjugates that target or bind to tumor-associated antigens (TAA) or tumor-specific antigens (TSA) (“collectively, “tumor antigens”). Antibodies targeting tumor antigens (“anti-tumor antibody”) are conjugated with very strong immune enhancer(s), recruiter(s) or solicitor(s) (hereinafter “immune enhancer”) analogous to potent toxins. The immune enhancer may be protein nano-particles, microbial antigens, viral particles or antigenic fragments thereof, or a combination of these. The immunoconjugates of the present embodiments are thus capable of specifically targeting cancer cells expressing tumor antigen and subsequently recruiting immune system components resulting in the cancer cell elimination.

### Immune Enhancers

The inventive composition contains a conjugate which comprises one or more immune enhancers. The immune enhancer is preferably an antigenic protein, glycoprotein or polysaccharide derived from a virus, bacteria, or other microorganism. In some embodiments, the immune enhancer is a viral antigen or an antigenic fragment/portion thereof or a bacterial antigen or a fragment thereof. In some embodiments, the immune enhancer is a viral antigen or an antigenic fragment/portion thereof or a bacterial antigen or a fragment thereof that can be recognized by autologous cytolytic T lymphocytes.

In some embodiments, the immune enhancer is a non-infectious, non-replicating virus or viral entity such as a viral particle or virus-like particle (VLP), or antigenic component thereof. Examples of viral entities include lentivirus, lambda virus and other bacteriophages.

The immune enhancer may be an antigenic protein (e.g., envelope protein or coat protein) or antigenic fragment thereof which contains at least one epitope. In some embodiments, the immune enhancer is a viral envelope glycoprotein. Examples of envelope glycoprotein include glycoprotein gp41, glycoprotein gp36, glycoprotein gp120, and fusogenic membrane glycoproteins.

In some embodiments, the immune enhancer is a tuberculosis (TB) antigen. The TB antigen may be selected from one or more of the following: ESAT-6, Ag85A, AG85B, MPT51, MPT64, CFP10, TB10.4, Mtb8.4, hspX, CFP6, Mtb12, Mtb9.9 antigens, Mtb32A, PstS-1, PstS-2, PstS-3, MPT 63, Mtb39, Mtb41, MPT83, 71-kDa, PPE 68, LppX, and antigenic portions thereof.

In some embodiments, the immune enhancer is viral hemagglutinin or antigenic fragment thereof. Viral hemagglutinin includes an influenza viral hemagglutinin protein such as influenza A viral hemagglutinin protein, influenza B viral hemagglutinin protein, or influenza C viral hemagglutinin protein. Influenza A is at least one member selected from the group consisting of H1, H2, H3, H5, H7 and H9.

In some embodiments, the immune enhancer is a viral particle or antigenic fragment thereof. The viral particle may be a lambda phage. The viral particle may be an adenovirus, adeno-associated virus (AAV), or lentivirus particle.

In some embodiments, the immune enhancer is a virus-like particle (VLP). In some embodiments, the immune enhancer is a virus-like particle of a bacteriophage. Examples of VLPs include, but are not limited to, the capsid proteins of Hepatitis B virus, measles virus, Sindbis virus, rotavirus, foot-and-mouth-disease virus, Norwalk virus the retroviral GAG protein, the retrotransposon Ty protein p1, the surface protein of Hepatitis B virus, human papilloma virus, RNA phages, Ty, fr-phage, GA-phage and Qβ-phage.

As will be readily apparent to those skilled in the art, the VLP of the present embodiments is not limited to any specific form. The particle can be synthesized chemically or through a biological process, which can be natural or non-natural. By way of example, this type of embodiment includes a virus-like particle or a recombinant form thereof. In some embodiments, the immune enhancer is a microbial antigen. The microbial antigen may be selected from the group consisting of a bacterial antigen, a mycobacterial antigen, a viral antigen, a fungal antigen, and a parasitic antigen. Preferred microbial antigens are lipopolysaccharides, hemagglutinins, Streptococcal antigens (e.g., Streptococcus pneumoniae polysaccharide type 4) and influenza antigens (e.g., influenza virus hemagglutinin).

In some embodiments, the immune enhancer is a bacterial antigen, which may be derived from a bacterial species selected from the group consisting of E. coli, Staphylococcus, Streptococcus, Pseudomonas, Clostridium difficile, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and Bacillus anthracis. The mycobacterial antigen may be derived from a mycobacterial species such as M. tuberculosis and M. leprae, but is not so limited. The bacterial antigen may be selected from Panton-Valentine Leukocidin (PVL) antigen of S. aureus, S. aureus Type 5, S. aureus Type 8, S. aureus 336, S. epidermidis PS1, S. epidermidis GP1, α-toxin, lipoteichoic acid (LTA) and microbial surface components recognizing adhesive matrix molecule (MSCRAMM) proteins. See U.S. Publication No. 20090074755, incorporated herein by reference in its entirety.

In some embodiments, the immune enhancer is a viral antigen, which may be derived from a viral species selected from the group consisting of HIV, Herpes simplex virus I, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS.

In some embodiments, the immune enhancer is a fungal antigen, which may be derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

In some embodiments, the immune enhancer is a parasitic antigen, which may be derived from a parasite species selected from the group consisting of Entamoeba, Trypanosoma cruzi, Fascioliasis, Leishmaniasis, Plasmodium, Onchocerciasis, Paragonimus, Trypanosoma brucei, Pneumocystis, Trichomonas vaginalis, Taenia, Hymenolepsis, Echinococcus, Schistosoma, Necator americanus, and Trichuris trichuria.

### Antibodies

The inventive compositions relate to immunoconjugates comprising an antibody. According to some embodiments, the antibody is an anti-tumor antibody. For example, immunoconjugates of the present embodiments contain antibodies to known cancer associated/specific antigen, or any target associated with undesirable or proliferating cells (e.g., prostate-specific antigen (PSA) for cancer and benign prostatic hypertrophy).

The antibody molecules can be of the various isotypes, including: IgG (e.g., IgG1, IgG2 (e.g., IgG2a, IgG2b), IgG3, IgG4), IgM, i.e., IgM/λ, IgA1, IgA2, IgD, or IgE. A preferred antibody molecule is an IgG isotype (e.g., IgG2). The antibody molecules can be full-length (e.g., an IgG1 or IgG4 antibody) or can include only an antigen-binding fragment (e.g., a Fab, F(ab′)₂, Fv or a single chain Fv fragment). In some embodiments, the antibody is an engineered antibody molecule, e.g., a fully human or a humanized antibody.

Any suitable antibody can be used in the inventive composition. In some embodiments, the immunoconjugates comprise an antibody (e.g., monoclonal antibody) to aspartyl (asparaginyl) beta-hydroxylase (HAAH) polypeptide. See e.g., U.S. Pat. No. 6,835,370, U.S. Pat. No. 6,783,758, U.S. Pat. No. 6,797,696, U.S. Pat. No. 6,812,206, U.S. Pat. No. 6,815,415, U.S. Pat. No. 6,835,370, U.S. Pat. No. 7,094,556, each of which is incorporated herein by reference in their entireties.

The antibody may have known therapeutic effects as a “naked” antibody or as an immunoconjugate such as an antibody conjugated to a radionuclide, toxin or other drug. In the case of the latter, the immune enhancers may serve as an additional conjugate to the known therapeutic immunoconjugate or may serve as a substitute for the conjugated radionuclide, toxin or other drug. That is, the antibody backbone of the immunoconjugate may be used to form the immunoconjugates with the immune enhancers disclosed herein. Examples of antibodies suitable for use in the present embodiments include, but are not limited to, the following: efalizumab, alefacept, infliximab, etanercept, basiliximab, daclizumab, muromonab, trastuzumab, ibritumomab, bevacizumab, cetuximab, rituximab, omalizumab, alemtuzumab, edrecolomab, panitumumab, and adalimumab.

The antibodies used in the present embodiments react immunologically with a tumor antigen and are thus anti-tumor antigen antibodies. Examples of tumor antigens include HER2, (EGFR) HER1, HER3, HER4, VEGFR, CD20, EpCAM, KIAA1815, LOC157378, FU20421, DSCD75, GPR160, GPCR41, SLC1A5, CEA, TRAIL, TRAIL-receptor 1, TRAIL-receptor 2, lymphotoxin-beta receptor, CCR4, CD19, CD22, CD28, CD33, CD40, CD80, CSF-1R, CTLA-4, fibroblast activation protein (FAP), hepsin, melanoma-associated chondroitin sulfate proteoglycan (MCSP), prostate-specific antigen (PSA), prostate-specific membrane antigen (PSMA), VEGF receptor 1, VEGF receptor 2, IGF1-R, TSLP-R, TIE-1, TIE-2, TNF-alpha, TNF like weak inducer of apoptosis (TWEAK), IL-1R, CEA, and IGF1-R. Among the most widely studied tumor antigens are melanoma associated antigens, prostate specific antigen (PSA), E6 and E7, carcinoembryonic antigen (CEA), p53, and gangliosides (e.g., GM2). Melanoma antigens including other MAGEs, MART-1, glycoprotein 100 (gp100), tyrosinase, BAGE, and GAGE. NY-ESO-1 may be targeted in the treatment of liver cancer. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies. In some embodiments, the tumor antigen is a breast cancer tumor antigen.

Tumor antigens that may be targeted with the antibody have been recited throughout the specification and include but are not limited to HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high molecular weight melanoma associated antigens recognized by antibody 9.2.27, HMFG-2, SM-3, B72.3, PR5C5, PR4D2, and the like. Other tumor antigens are described in U.S. Pat. No. 5,776,427, incorporated by reference herein in its entirety.

Tumor antigens can be classified in a variety of ways. Tumor antigens include antigens encoded by genes that have undergone chromosomal alteration. Many of these antigens are found in lymphoma and leukemia. Even within this classification, antigens can be characterized as those that involve activation of quiescent genes. These include BCL-1 and IgH (Mantel cell lymphoma), BCL-2 and IgH (Follicular lymphoma), BCL-6 (Diffuse large B-cell lymphoma), TAL-1 and TCR.delta. or SIL (I-cell acute lymphoblastic leukemia), c-MYC and IgH or IgL (Burkitt lymphoma), MUN/IRF4 and IgH (Myeloma), PAX-5 (BSAP) (Immunocytoma).

Other tumor antigens that involve chromosomal alteration and thereby create a novel fusion gene and/or protein include RARoa, PML, PLZF, NPM or NuM4 (Acute promyelocytic leukemia), BCR and ABL (Chronic myeloid/acute lymphoblastic leukemia), MLL (HRX) (Acute leukemia), E2A and PBX or HLF (B-cell acute lymphoblastic leukemia), NPM, ALK (Anaplastic large cell leukemia), and NPM, MLF-1 (Myelodysplastic syndrome/acute myeloid leukemia).

Other tumor antigens are specific to a tissue or cell lineage. These include cell surface proteins such as CD20, CD22 (Non-Hodgkin's lymphoma, B-cell lymphoma, Chronic lymphocytic leukemia (CLL)), CD52 (B-cell CLL), CD33 (Acute myelogenous leukemia (AML)), CD 10(gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (Epithelial and lymphoid malignancies), Human leukocyte antigen (HLA)-DR, HLA-DP, and HLA-DQ (Lymphoid malignancies), RCAS1 (Gynecological carcinomas, bilary adenocarcinomas and ductal adenocarcinomas of the pancreas), and Prostate specific membrane antigen (Prostate cancer).

Tissue- or lineage-specific tumor antigens also include epidermal growth factor receptors (high expression) such as EGFR (HER1 or erbB1) and EGFRvIII (Brain, lung, breast, prostate and stomach cancer), erbB2 (HER2 or HER2/neu) (Breast cancer and gastric cancer), erbB3 (HER3) (Adenocarcinoma), and erbB4 (HER4) (Breast cancer).

Tissue- or lineage-specific tumor antigens also include cell-associated proteins such as Tyrosinase, Melan-A/MART-1, tyrosinase related protein (TRP)-1/gp75 (Malignant melanoma), Polymorphic epithelial mucin (PEM) (Breast tumors), and Human epithelial mucin (MUC1) (Breast, ovarian, colon and lung cancers).

Tissue- or lineage-specific tumor antigens also include secreted proteins such as Monoclonal immunoglobulin (Multiple myeloma and plasmacytoma), Immunoglobulin light chains (Multiple Myeloma), alpha.-fetoprotein (Liver carcinoma), Kallikreins 6 and 10 (Ovarian cancer), Gastrin-releasing peptide/bombesin (Lung carcinoma), and Prostate specific antigen (Prostate cancer).

Still other tumor antigens are cancer testis (CT) antigens that are expressed in some normal tissues such as testis and in some cases placenta. Their expression is common in tumors of diverse lineages and as a group the antigens form targets for immunotherapy. Examples of tumor expression of CT antigens include MAGE-A1, -A3, -A6, -A12, BAGE, GAGE, HAGE, LAGE-1, NY-ESO-1, RAGE, SSX-1, -2, -3, -4, -5, -6, -7, -8, -9, HOM-TES-14/SCP-1, HOM-TES-85 and PRAME. Still other examples of CT antigens and the cancers in which they are expressed include SSX-2, and -4 (Neuroblastoma), SSX-2 (HOM-MEL-40), MAGE, GAGE, BAGE and PRAME (Malignant melanoma), HOM-TES-14/SCP-1 (Meningioma), SSX-4 (Oligodendrioglioma), HOM-TES-14/SCP-1, MAGE-3 and SSX-4 (Astrocytoma), SSX member (Head and neck cancer, ovarian cancer, lymphoid tumors, colorectal cancer and breast cancer), RAGE-1, -2, -4, GAGE-1-2, -3, -4, -5, -6, -7 and -8 (Head and neck squamous cell carcinoma (HNSCC)), HOM-TES14/SCP-1, PRAME, SSX-1 and CT-7 (Non-Hodgkin's lymphoma), and PRAME (Acute lymphoblastic leukemia (ALL), acute myelogenous leukemia (AML) and chronic lymphocytic leukemia (CLL)).

Other tumor antigens are not specific to a particular tissue or cell lineage. These include members of the carcinoembryonic antigen (CEA) CD66a, CD66b, CD66c, CD66d and CD66e. These antigens can be expressed in many different malignant tumors and can be targeted by immunotherapy.

Still other tumor antigens are viral proteins and these include Human papilloma virus protein (cervical cancer), and EBV-encoded nuclear antigen (EBNA)-1 (lymphomas of the neck and oral cancer).

Still other tumor antigens are mutated or aberrantly expressed molecules such as but not limited to CDK4 and beta-catenin (melanoma).

In some embodiments, the antigen is a tumor antigen. The tumor antigen may be selected from the group consisting of MART-1/Melan-A, gp100, adenosine deaminase-binding protein (ADAbp), FAP, cyclophilin b, colorectal associated antigen (CRC)-C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, and CD20. The tumor antigen may also be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE A11, MAGE-A12, MAGE-Xp2 (MAGE B2), MAGE-Xp3 (MAGE-B3), MAGE-Xp4 (MAGE-B4), MAGE-C1, MAGE-C2, MAGE-C3, MAGE-C4, MAGE-C5). In still another embodiment, the tumor antigen is selected from the group consisting of GAGE -1, GAGE-2, GAGE-3, GAGE-4, GAGE-5, GAGE-6, GAGE-7, GAGE-8, GAGE-9. And in yet a further embodiment, the tumor antigen is selected from the group consisting of BAGE, RAGE, LAGE-1, NAG, GnT-V, MUM-1, CDK4, tyrosinase, p53, MUC family, HER2/neu, p21 ras, RCAS 1, α-fetoprotein, E-cadherin, α-catenin, β-catenin, .gamma.-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Cancer or tumor antigens can also be classified according to the cancer or tumor they are associated with (i.e., expressed by). Cancers or tumors associated with tumor antigens include acute lymphoblastic leukemia (etv6; am11; cyclophilin b), B cell lymphoma (Ig-idiotype); Burkitt's (Non-Hodgkin's) lymphoma (CD20); glioma (E-cadherin; α-catenin; β-catenin; .gamma.-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)-C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkin's lymphoma (Imp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21 ras), non-small cell lung carcinoma (HER2/neu; c-erbB-2), nasopharyngeal cancer (Imp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins and non-infectious particles), testicular cancer (NY-ESO-1), T cell leukemia (HTLV-1 epitopes), and melanoma (Melan-A/MART-1; cdc27; MAGE-3; p21ras; gp100^{Pmc117}).

Still other cancer antigens are listed in Table 1.

TABLE 1
Tumor-specific antigens

| | | | Lymphocyte | |
|---|---|---|---|---|
| Gene | Peptide | Position | Stimulation Method | References |
| BAGE-1 | AARAVFLAL | 2-10 | autologous tumor cells | Boel, 1995 |
| GAGE-1, 2, 8 | YRPRPRRY | 9-16 | autologous tumor cells | Van den Eynde, 1995 |
| GAGE-3, 4, 5, | YYWPRPRRY | 10-18 | autologous tumor cells | De Backer, 1999 |
| 6, 7 | | | | |
| GnTV^{f} | VLPDVFIRC(V) | intron | autologous tumor cells | Guilloux, 1996 |
| HERV-K-MEL | MLAVISCAV | 1-9 | autologous tumor cells | Schiavetti, 2002 |
| KK-LC-1 | RQKRILVNL | 76-84 | autologous tumor cells | Fukuyama, 2006 |
| | EYSKECLKEF | 499-508 | peptide | Monji, 2004 |
| | EYLSLSDKI | 770-778 | peptide | Monji, 2004 |
| LAGE-1 | MLMAQEALAFL | ORF2 | autologous tumor cells | Aarnoudse, 1999 |
| | | (1-11) | | |
| | SLLMWITQC | 157-165 | peptide | Rimoldi, 2000 |
| | LAAQERRVPR | ORF2 | autologous tumor cells | Wang, 1998 |
| | | (18-27) | | |
| | ELVRRILSR | 103-111 | adenovirus-dendritic | Sun, 2006 |
| | | | cells | |
| | APRGVRMAV | ORF2 | adenovirus-APC | Slager, 2004b |
| | | (46-54) | | |
| | SLLMWITQCFLPVF | 157-170 | peptide | Zeng, 2001 |
| | QGAMLAAQERRVPRAAE | ORF2 | protein | Slager, 2004a |
| | VPR | (14-33) | | |
| | AADHRQLQLSISSCLQQ | 139-156 | protein | Jager, 2000 |
| | L | | | |
| | CLSRRPWKRSWSAGSCP | ORF2 | peptide | Slager, 2003 |
| | GMPHL | (81-102) | | |
| | CLSRRPWKRSWSAGSCP | ORF2 | peptide | Slager, 2003 |
| | GMPHL | (81-102) | | |
| | ILSRDAAPLPRPG | 108-120 | autologous tumor cells | Wang, 2004 |
| | AGATGGRGPRGAGA | 37-50 | protein | Hasegawa, 2006 |
| MAGE-A1 | EADPTGHSY | 161-169 | autologous tumor cells | Traversari, 1992 |
| | KVLEYVIKV | 278-286 | peptide | Ottaviani, 2005 |
| | | | | Pascolo, 2001 |
| | SLFRAVITK | 96-104 | poxvirus-dendritic cells^{c} | Chaux, 1999a |
| | EVYDGREHSA | 222-231 | poxvirus-dendritic cells | Chaux, 1999a |
| | RVRFFFPSL | 289-298 | poxvirus-dendritic cells | Luiten, 2000a |
| | EADPTGHSY | 161-169 | poxvirus-dendritic cells | Luiten, 2000b |
| | REPVTKAEML | 120-129 | autologous tumor cells | Tanzarella, 1999 |
| | DPARYEFLW | 258-266 | poxvirus-dendritic cells | Chaux, 1999a |
| | ITKKVADLVGF | 102-112 | ALVAC-dendritic cells | Corbière, 2004 |
| | SAFPTTINF | 62-70 | poxvirus-dendritic cells | Chaux, 1999a |
| | SAYGEPRKL | 230-238 | poxvirus-dendritic cells | Chaux, 1999a |
| | SAYGEPRKL | 230-238 | autologous tumor cells | van der Bruggen, 1994a |
| | TSCILESLFRAVITK | 90-104 | peptide | Wang, 2007 |
| | PRALAETSYVKVLEY | 268-282 | peptide | Wang, 2007 |
| | FLLLKYRAREPVTKAE | 112-127 | protein | Chaux, 1999b |
| | EYVIKVSARVRF | 281-292 | protein | Chaux, 2001 |
| MAGE-A2 | YLQLVFGIEV | 157-166 | peptide | Kawashima, 1998 |
| | EYLQLVFGI | 156-164 | peptide | Tahara, 1999 |
| | REPVTKAEML | 127-136 | autologous tumor cells | Tanzarella, 1999 |
| | EGDCAPEEK | 212-220 | lentivirus-dendritic cells | Breckpot, 2004 |
| | LLKYRAREPVTKAE | 121-134 | protein | Chaux, 1999b |
| MAGE-A3 | EVDPIGHLY | 168-176 | autologous tumor cells | Gaugler, 1994 |
| | FLWGPRALV^{d} | 271-279 | peptide | van der Bruggen, 1994b |
| | KVAELVHFL | 112-120 | peptide | Kawashima, 1998 |
| | TFPDLESEF | 97-105 | peptide | Oiso, 1999 |
| | VAELVHFLL | 113-121 | peptide | Miyagawa, 2006 |
| | MEVDPIGHLY | 167-176 | adeno-dendritic cells | Bilsborouh, 2002 |
| | EVDPIGHLY | 168-176 | poxvirus-dendritic cells | Schultz, 2001 |
| | REPVTKAEML | 127-136 | autologous tumor cells | Tanzarella, 1999 |
| | AELVHFLLLⁱ | 114-122 | adeno-dendritic cells | Schultz, 2002 |
| | MEVDPIGHLY | 167-176 | peptide | Herman, 1996 |
| | WQYFFPVIF | 143-151 | retrovirus-dendritic cells^{h} | Russo, 2000 |
| | EGDCAPEEK | 212-220 | lentivirus-dendritic cells | Breckpot, 2004 |
| | KKLLTQHFVQENYLEY | 243-258 | protein | Schultz, 2000 |
| | KKLLTQHFVQENYLEY | 243-258 | peptide | Schultz, 2000 |
| | ACYEFLWGPRALVETS | 267-282 | protein | Zhang, 2003 |
| | RKVAELVHFLLLKYR | 111-125 | peptide | Cesson, 2010 |
| | VIFSKASSSLQL | 149-160 | peptide | Kobayashi, 2001 |
| | VIFSKASSSLQL | 149-160 | peptide | Kobayashi, 2001 |
| | VFGIELMEVDPIGHL | 161-175 | peptide | Cesson, 2010 |
| | GDNQIMPKAGLLIIV | 191-205 | peptide | Consogno, 2003 |
| | TSYVKVLHHMVKISG | 281-295 | protein | Manici, 1999 |
| | RKVAELVHFLLLKYRA | 111-126 | protein | Chaux, 1999b |
| | FLLLKYRAREPVTKAE | 119-234 | protein | Chaux, 1999b |
| MAGE-A4 | EVDPASNTY^{j} | 169-177 | peptide after tetramer | Kobayashi, 2003 |
| | | | sorting | |
| | GVYDGREHTV | 230-239 | adeno-dendritic cells | Duffour, 1999 |
| | NYKRCFPVI | 143-151 | peptide | Miyahara, 2005 |
| | | | | Ottaviani, 2006 |
| | SESLKMIF | 156-163 | poxvirus-dendritic cells | Zhang, 2002 |
| MAGE-A6 | MVKISGGPR | 290-298 | autologous tumor cells | Zorn, 1999 |
| | EVDPIGHVY | 168-176 | autologous tumor cells | Benlalam, 2003 |
| | REPVTKAEML | 127-136 | autologous tumor cells | Tanzarella, 1999 |
| | EGDCAPEEK | 212-220 | lentivirus-dendritic cells | Breckpot, 2004 |
| | ISGGPRISY | 293-301 | autologous tumor cells | Vantomme, 2003 |
| | LLKYRAREPVTKAE | 121-134 | protein | Chaux, 1999b |
| MAGE-A9 | ALSVMGVYV | 223-231 | peptide | Oehlrich, 2005 |
| MAGE-A10 | GLYDGMEHL^{l} | 254-262 | autologous tumor cells | Huang, 1999 |
| | DPARYEFLW | 290-298 | poxvirus-dendritic cells | Chaux, 1999a |
| MAGE-A12 | FLWGPRALV^{e} | 271-279 | peptide | van der Bruggen, 19994b |
| | VRIGHLYIL | 170-178 | autologous tumor cells | Heidecker, 2000 |
| | | | | Panelli, 2000 |
| | EGDCAPEEK | 212-220 | lentivirus-dendritic cells | Breckpot, 2004 |
| | REPFTKAEMLGSVIR | 127-141 | peptide | Wang, 2007 |
| | AELVHFLLLKYRAR | 114-127 | protein | Chaux, 1999b |
| MAGE-C1 | SSALLSIFQSSPE | 137-149 | peptide | Nuber, 2010 |
| | SFSYTLLSL | 450-458 | peptide | Nuber, 2010 |
| | VSSFFSYTL | 779-787 | peptide | Nuber, 2010 |
| MAGE-C2 | LLFGLALIEV | 191-200 | autologous tumor cells | Ma, 2004 |
| | ALKDVEERV | 336-344 | autologous tumor cells | Ma, 2004 |
| | SESIKKKVL | 307-315 | autologous tumor cells | Godelaine, 2007 |
| mucin^{k} | PDTRPAPGSTAPPAHGV | | transfected B cells | Jerome, 1993 |
| | TSA | | | |
| NA88-A | QGQHFLQKV | | tumor-infiltrating | Moreau-Aubry, 2000 |
| | | | lymphocytes | |
| NY-ESO-1/ | SLLMWITQC | 157-165 | autologous tumor cells | Jager, 1998 |
| LAGE-2 | | | | Chen, 2000 |
| | | | | Valmori, 2000 |
| | MLMAQEALAFL | ORF2 | autologous tumor cells | Aarnoudse, 1999 |
| | | (1-11) | | |
| | ASGPGGGAPR | 53-62 | autologous tumor cells | Wang, 1998 |
| | LAAQERRVPR | ORF2 | autologous tumor cells | Wang, 1998 |
| | | (18-27) | | |
| | TVSGNILTIR | 127-136 | mRNA-transfected cells | Matsuzaki, 2008 |
| | APRGPHGGAASGL | 60-72 | peptide | Ebert, 2009 |
| | MPFATPMEA | 94-102 | autologous tumor cells | Benlalam, 2003 |
| | KEFTVSGNILTI | 124-135 | mRNA-transfected cells | Knights, 2009 |
| | MPFATPMEA | 94-102 | adenovirus-APC | Jäger, 2002 |
| | LAMPFATPM | 92-100 | adenovirus-PBMC | Gnjatic, 2000 |
| | ARGPESRLL | 80-88 | adenovirus-PBMC^{d} | Gnjatic, 2000 |
| | SLLMWITQCFLPVF | 157-170 | peptide | Zeng, 2001 |
| | LLEFYLAMPFATPMEAE | 87-111 | peptide | Mandic, 2005 |
| | LARRSLAQ | | | |
| | LLEFYLAMPFATPMEAE | 87-111 | peptide | Mandic, 2005 |
| | LARRSLAQ | | | |
| | EFYLAMPFATPM | 89-100 | protein | Chen, 2004 |
| | PGVLLKEFTVSGNILTI | 119-143 | peptide | Ayyoub, 2010 |
| | RLTAADHR | | | |
| | RLLEFYLAMPFA | 86-97 | protein | Chen, 2004 |
| | QGAMLAAQERRVPRAAE | ORF2 | protein | Slager, 2004a |
| | VPR | (14-33) | | |
| | PFATPMEAELARR | 95-107 | peptide | Mizote, 2010 |
| | PGVLLKEFTVSGNILTI | 119-138 | peptide and protein | Jager, 2000 |
| | RLT | | | Zarour, 2000 |
| | VLLKEFTVSG | 121-130 | peptide | Zeng, 2000 |
| | AADHRQLQLSISSCLQQ | 139-156 | protein | Jager, 2000 |
| | L | | | |
| | LLEFYLAMPFATPMEAE | 87-111 | peptide | Mandic, 2005 |
| | LARRSLAQ | | | |
| | LKEFTVSGNILTIRL | 123-137 | protein | Bioley, 2009 |
| | PGVLLKEFTVSGNILTI | 119-143 | peptide | Zarour, 2002 |
| | RLTAADHR | | | |
| | LLEFYLAMPFATPMEAE | 87-111 | peptide | Mandic, 2005 |
| | LARRSLAQ | | | |
| | KEFTVSGNILT | 123-134 | peptide | Mizote, 2010 |
| | LLEFYLAMPFATPM | 87-100 | peptide | Mizote, 2010 |
| | AGATGGRGPRGAGA | 37-50 | protein | Hasegawa, 2006 |
| SAGE | LYATVIHDI | 715-723 | peptide | Miyahara, 2005 |
| Sp17 | ILDSSEEDK | 103-111 | protein | Chiriva-Internati, 2003 |
| SSX-2 | KASEKIFYV | 41-49 | autologous tumor cells | Ayyoub, 2002 |
| | EKIQKAFDDIAKYFSK | 19-34 | peptide | Ayyoub, 2004a |
| | WEKMKASEKIFYVYMKR | 37-54 | peptide | Ayyoub, 2005a |
| | K | | | |
| | KIFYVYMKRKYEAMT | 45-59 | peptide | Neumann, 2004 |
| | KIFYVYMKRKYEAM | 45-58 | protein | Ayyoub, 2004b |
| SSX-4 | INKTSGPKRGKHAWTHR | 151-170 | peptide | Ayyoub, 2005b |
| | LRE | | | |
| | YFSKKEWEKMKSSEKIV | 31-50 | peptide | Ayyoub, 2005b |
| | YVY | | | |
| | MKLNYEVMTKLGFKVTL | 51-70 | peptide | Valmori, 2006 |
| | PPF | | | |
| | KHAWTHRLRERKQLVVY | 161-180 | peptide | Valmori, 2006 |
| | EEI | | | |
| | LGFKVTLPPFMRSKRAA | 61-80 | peptide | Ayyoub, 2005b |
| | DFH | | | |
| | KSSEKIVYVYMKLNYEV | 41-60 | peptide | Ayyoub, 2005b |
| | MTK | | | |
| | KHAWTHRLRERKQLVVY | 161-180 | peptide | Valmori, 2006 |
| | EEI | | | |
| TAG-1 | SLGWLFLLL | 78-86 | peptide | Adair, 2008 |
| | LSRLSNRLL | 42-50 | peptide | Adair, 2008 |
| TAG-2 | LSRLSNRLL | 42-50 | peptide | Adair, 2008 |
| TRAG-3 | CEFHACWPAFTVLGE | 34-48 | peptide | Janjic, 2006 |
| | CEFHACWPAFTVLGE | 34-48 | peptide | Janjic, 2006 |
| | CEFHACWPAFTVLGE | 34-48 | peptide | Janjic, 2006 |
| TRP2-INT2^{g} | EVISCKLIKR | intron 2 | autologous tumor cells | Lupetti, 1998 |
| XAGE-1b | CATWKVICKSCISQTPG | 33-49 | autologous tumor cells | Shimono, 2007 |

The antibody molecule may be a fully human, chimeric or humanized monoclonal antibody, examples of which include huN901, huMy9-6 (ATCC PTA-4786, deposited on Nov. 15, 2002, American Type Culture Collection, 10801 University Boulevard, Manassas, Va. 20110-2209), huB4, huC242, trastuzumab, bivatuzumab, sibrotuzumab, and rituximab. The antibody may be the huN901 humanized monoclonal antibody or the huMy9-6 humanized monoclonal antibody. Other humanized monoclonal antibodies are known in the art and can be used in connection with the inventive composition.

In some embodiments, the antibody molecule of the immunoconjugates of the present invention is an anti-vascular endothelial growth factor (VEGF) antibody. For example, the anti-VEGF antibody molecule may be the monoclonal antibody bevacizumab (Avastin®), which is approved to treat a number of cancers. An immunoconjugate comprising bevacizumab (Avastin®) may be used for the treatment of the following conditions: colon cancer; rectal cancer; non-squamous, non-small cell lung cancer; breast cancer; glioblastoma brain cancer; renal cell carcinoma (a type of kidney cancer).

In some embodiments, the antibody molecule of the immunoconjugates of the present invention is an anti-CD20 antibody. For example, the anti-CD20 antibody molecule may be tositumomab (Bexxar®), a murine IgG2a lambda monoclonal antibody directed against the CD20 antigen, which is found on the surface of normal and malignant B lymphocytes. Tositumomab is produced in an antibiotic-free culture of mammalian cells and is composed of two murine gamma 2a heavy chains of 451 amino acids each and two lambda light chains of 220 amino acids each. Ibritumomab (Zevalin®) and rituximab (Rituxan®) are other anti-CD20 antibodies suitable for the present embodiments. An immunoconjugate comprising an anti-CD20 antibody may be used for the treatment of the following conditions: B cell non-Hodgkin's lymphoma, a lymphoproliferative disorder and thus affects the lymphatic system.

In some embodiments, the antibody molecule of the immunoconjugates of the present invention is an anti-CD33 antibody.

In some embodiments, the antibody molecule of the immunoconjugates of the present invention is an anti-HER2 antibody. For example, the anti-HER2 antibody molecule may be trastuzumab (Herceptin®). An immunoconjugate comprising an anti-HER2 antibody may be used for the treatment of breast cancer.

In some embodiments, the antibody molecule of the immunoconjugates of the present invention is an anti-epidermal growth factor receptor (EGFR) antibody. For example, the anti-EGFR antibody molecule may be Erbitux® (cetuximab), an epidermal growth factor receptor (EGFR) antagonist. An immunoconjugate comprising Erbitux® (cetuximab) may be used for the treatment of of locally or regionally advanced squamous cell carcinoma of the head and neck. Thus, in some embodiments, the antibody molecule binds EGFR, preferably a monoclonal antibody selected from the group consisting of: cetuximab, panitumumab, zalutumumab, nimotuzumab, necitumumab and matuzumab.

In some embodiments, the antibody molecule of the immunoconjugates of the present invention is an anti-CTLA-4 (cytotoxic T lymphocyte-associated antigen 4) antibody. For example, the anti-CTLA-4 antibody molecule may be YERVOY™ (ipilimumab). An immunoconjugate comprising an anti-CTLA-4 antibody may be used for the treatment of melanoma.

In some embodiments, the immunoconjugates of the present embodiments comprise one or more of the following antibody molecules: an Anti-CD137 antibody; an Anti-CS1 antibody (e.g., Elotuzumab); an Anti-PD-L1 antibody; an Anti-PD1 antibody; and Anti-CD19 antibody; and an Anti-CXCR4 antibody.

### Immunoconjugates

In some embodiments, the immune enhancer is chemically conjugated to the antibody. In some embodiments, the antibody and a first immune enhancer (fIE) may be chemically conjugated to a second immune enhancer (sIE). Examples of such embodiments may are described by the following formulas:

(Antibody)-(fIE)-(sIE) and

(fIE)-(Antibody)-(sIE).

For example, an anti-HAAH antibody may be chemically conjugated to a non-replicating lambda virus (or fragment thereof), which is also conjugated to one or more TB antigens resulting in an immunoconjugate with the following formula:

(Anti-HAAH)-(Non-Replicating Lambda Virus)-(TB Antigens).

This entity is capable of binding cancer cells expressing HAAH on their surface and promoting the subsequent recruitment of immune system components and systemic immune response. Use of the immunoconjugates of the present embodiments thus allows for the benefits of cancer cell elimination, specifically, without any toxic effect common with the use of toxins.

In some embodiments, where the immune enhancer is a virus, the virus is engineered to express the antibody (e.g., single chain fragment) and/or another immune enhancer (e.g., TB antigen) on its surface.

### Compositions

The compositions of the present embodiments comprises a therapeutically effective amount of a conjugate comprising an antibody chemically coupled to an immune enhancer. A “therapeutically effective amount” means an amount sufficient to show a meaningful benefit in an individual, e.g., promoting at least one aspect of tumor cell cytotoxicity, or treatment, healing, prevention, or amelioration of other relevant medical condition(s) associated with a particular cancer. Therapeutically effective amounts may vary depending upon the biological effect desired in the individual, condition to be treated, and/or the specific characteristics of the conjugate, and the individual. Thus, in accordance with the methods described herein, the attending physician (or other medical professional responsible for administering the composition) will typically decide the amount of the composition with which to treat each individual patient. The concentration of the conjugate in the inventive composition desirably is about 0.1 mg/mL to about 5 mg/mL (e.g., about 0.5 mg/mL, about 2 mg/mL, or about 5 mg/mL). In a preferred embodiment, the concentration of the conjugate in the inventive composition is about 1 mg/mL or higher (e.g., about 2 mg/mL or higher, about 3 mg/mL or higher, or about 4 mg/mL or higher). Most preferably, the concentration of the conjugate in the inventive composition is about 5 mg/mL. While compositions comprising at least 1 mg/mL of the conjugate are particularly preferred, conjugate concentrations of less than 1 mg/mL (e.g., concentrations of about 0.1 mg/mL to about 0.9 mg/mL) also can be stably maintained in the inventive composition, and thus are within the scope of the invention. Compositions comprising greater than 1 mg/mL of the conjugate molecule are advantageous for clinical and commercial use, in that such concentrations enable single doses of the composition to be prepared in a more convenient (i.e., smaller) volume for administration,

The inventive composition desirably is formulated to be acceptable for pharmaceutical use, such as, for example, administration to a human host in need thereof. To this end, the conjugate molecule preferably is formulated into a composition comprising a physiologically acceptable carrier (e.g., excipient or diluent). Physiologically acceptable carriers are well known and are readily available, and include buffering agents, anti-oxidants, bacteriostats, salts, and solutes that render the formulation isotonic with the blood or other bodily fluid of the human patient, and aqueous and non-aqueous sterile suspensions that can include suspending agents, solubilizers, thickening agents, stabilizers (e.g., surfactants), and preservatives. The choice of carrier will be determined, at least in part, by the location of the target tissue and/or cells, and the particular method used to administer the composition. Examples of suitable carriers and excipients for use in drug conjugate formulations are known in the art.

### Methods of Treatment

The invention provides methods that relate to a novel therapeutic strategy for the treatment of cancer. In particular, the method comprises administration of one or more immunoconjugates of the present embodiments or a pharmaceutical composition comprising such one or more immunoconjugates admixed with at least one pharmaceutically acceptable excipient.

The immunoconjugates of the present embodiments are useful to treat certain cancers. In some embodiments, the cancer is a hematopoietic cancer. In some embodiments, the hematopoietic cancer is selected from the group consisting of leukemia, lymphoma, and myeloma.

In some embodiments, the hematopoietic cancer is of lymphoid lineage. In some embodiments, the hematopoietic cancer is of lymphoid lineage is selected from leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma.

In some embodiments, the hematopoietic cancer is of myeloid lineage. In some embodiments, the hematopoietic cancer of myeloid lineage is selected from acute myelogenous leukemia, chronic myelogenous leukemia, multiple myelogenous leukemia, myelodysplastic syndrome and promyelocytic leukemia.

In some embodiments, the cancer is a non-hematopoietic cancer. In some embodiments, the cancer is a solid tumor. In some embodiments, the cancer is selected from pancreatic cancer; bladder cancer; colorectal cancer; breast cancer; prostate cancer; renal cancer; hepatocellular cancer; lung cancer; ovarian cancer; cervical cancer; gastric cancer; esophageal cancer; head and neck cancer; melanoma; neuroendocrine cancers; CNS cancers; brain tumors; bone cancer; and soft tissue sarcoma. In some embodiments it is lung cancer (non-small cell lung cancer, small-cell lung cancer), colon cancer, CNS cancer, melanoma, ovarian cancer, renal cancer, prostate cancer or breast cancer.

According to some embodiments, methods are provided for killing a cell in a human comprising administering to the human a composition comprising a therapeutically effective amount of a conjugate comprising an antibody chemically coupled to an immune enhancer.

The inventive method involves administering the inventive composition to a human. Ideally, the inventive method is used to target and kill cells affected by a disease, particularly a disease associated with elevated levels of cellular proliferation, such as cancer. Thus, in this regard, the inventive method preferably is used to kill tumor cells in a human, thereby resulting in the prevention, amelioration, and/or cure of the cancer.

While any suitable means of administering the composition to a human can be used within the context of the invention, typically and preferably the inventive composition is administered to a human via injection, and most preferably via infusion. By the term “injection,” it is meant that the composition is forcefully introduced into a target tissue of the human. By the term “infusion,” it is meant that the composition is introduced into a tissue, typically and preferably a vein, of the human. The composition can be administered to the human by any suitable route, but preferably is administered to the human intravenously or intraperitoneally. When the inventive method is employed to kill tumor cells, however, intratumoral administration of the inventive composition is particularly preferred. When the inventive composition is administered by injecting, any suitable injection device can be used to administer the composition directly to a tumor. For example, the common medical syringe can be used to directly inject the composition into a subcutaneous tumor. Alternatively, the composition can be topically applied to the tumor by bathing the tumor in the inventive liquid composition. Likewise, the tumor can be perfused with the inventive composition over a period of time using any suitable delivery device, e.g., a catheter. While less preferred, other routes of administration can be used to deliver the composition to the human. Indeed, although more than one route can be used to administer the inventive composition, a particular route can provide a more immediate and more effective reaction than another route. For example, while not particularly preferred, the inventive composition can be applied or instilled into body cavities, absorbed through the skin, inhaled, administered subcutaneously, intradermaly, intranasaly, or administered parenterally via, for instance, intramuscular or intraarterial administration. Preferably, the inventive composition parenterally administered to a human is specifically targeted to particular cells, e.g., cancer cells.

As described herein, the conjugate comprises an antibody, which may be a fully human, chimeric or humanized monoclonal antibody, such as an anti-tumor antibody. Suitable antibodies include, for example, trastuzumab, bivatuzumab, sibrotuzumab, and rituximab. When compositions comprising such conjugates are employed in the inventive method, the antibody targets the conjugate to a desired cell (e.g., a tumor cell) through interactions with antigens (e.g., tumor-specific antigens) expressed at the surface of the cell (e.g., tumor cell). Tumor-specific antigens have been extensively described in the prior art for a variety of tumors, including, for example, epithelial cancers (e.g., MUC1), and breast and ovarian cancer (e.g., HER2/neu), and as described herein.

For the purposes of human administration, the inventive liquid composition described herein may be administered (e.g., infused) directly to a human, or diluted with a suitable diluent immediately prior to administration. Suitable diluents are known in the art and include D5W and normal saline (NS). Dilutions of 1:1, 1:2, 1:3, or more (e.g., 1:5, 1:10, or even 1:50) with suitable diluents are possible. Dilution of the inventive composition desirably does not reduce the concentration of the conjugate molecule in the composition below about 0.1 mg/mL. Upon diluting the inventive liquid composition, the previously described concentrations of each of the components (e.g., buffering agent, surfactant, and sodium chloride) of the composition are correspondingly reduced.

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

The term “antibody,” as used herein, refers to any immunoglobulin, any antigen-binding portion, any immunoglobulin fragment, such as Fab, F(ab′)₂, dsFv, sFv, diabodies, and triabodies, or immunoglobulin chimera, which can bind to an antigen on the surface of a cell (e.g., which contains a complementarity determining region (CDR)). Any suitable antibody can be used in the inventive composition. One of ordinary skill in the art will appreciate that the selection of an appropriate antibody will depend upon the cell population to be targeted. In this regard, the type and number of cell surface molecules (i.e., antigens) that are selectively expressed in a particular cell population (typically and preferably a diseased cell population) will govern the selection of an appropriate antibody for use in the inventive composition. Cell surface expression profiles are known for a wide variety of cell types, including tumor cell types, or, if unknown, can he determined using routine molecular biology and histochemistry techniques.

The antibody can be polyclonal or monoclonal, but is most preferably a monoclonal antibody. As used herein, “polyclonal” antibodies refer to heterogeneous populations of antibody, typically contained in the sera of immunized animals. “Monoclonal” antibodies refer to homogenous populations of antibody molecules that are specific to a particular antigen. Monoclonal antibodies are typically produced by a single clone of B lymphocytes (“B cells”). Monoclonal antibodies may be obtained using a variety of techniques known to those skilled in the art, including standard hybridoma technology. In brief, the hybridoma method of producing monoclonal antibodies typically involves injecting any suitable animal, typically and preferably a mouse, with an antigen (i.e., an “immunogen”). The animal is subsequently sacrificed, and B cells isolated from its spleen are fused with human myeloma cells. A hybrid cell is produced (i.e., a “hybridoma”), which proliferates indefinitely and continuously secretes high titers of an antibody with the desired specificity in vitro. Any appropriate method known in the art can be used to identify hybridoma cells that produce an antibody with the desired specificity. Such methods include, for example, enzyme-linked immunosorbent assay (ELISA), Western blot analysis, and radioimmunoassay. The population of hybridomas is screened to isolate individual clones, each of which secrete a single antibody species to the antigen. Because each hybridoma is a clone derived from fusion with a single B cell, all the antibody molecules it produces are identical in structure, including their antigen binding site and isotype. Monoclonal antibodies also may be generated using other suitable techniques including EBV-hybridoma technology or bacteriophage vector expression systems. To prepare monoclonal antibody fragments, recombinant methods typically are employed.

The monoclonal antibody can be isolated from or produced in any suitable animal, but is preferably produced in a mammal, more preferably a mouse, and most preferably a human. Methods for producing an antibody in mice are well known to those skilled in the art and are described herein. With respect to human antibodies, one of ordinary skill in the art will appreciate that polyclonal antibodies can be isolated from the sera of human subjects vaccinated or immunized with an appropriate antigen. Alternatively, human antibodies can be generated by adapting known techniques for producing human antibodies in non-human animals such as mice.

While being the ideal choice for therapeutic applications in humans, human antibodies, particularly human monoclonal antibodies, typically are more difficult to generate than mouse monoclonal antibodies. Mouse monoclonal antibodies, however, induce a rapid host antibody response when administered to humans, which can reduce the therapeutic or diagnostic potential of the antibody-drug conjugate. To circumvent these complications, a monoclonal antibody preferably is not recognized as “foreign” by the human immune system. To this end, phage display can be used to generate the antibody. In this regard, phage libraries encoding antigen-binding variable (V) domains of antibodies can be generated using standard molecular biology and recombinant DNA techniques. Phage encoding a variable region with the desired specificity are selected for specific binding to the desired antigen, and a complete human antibody is reconstituted comprising the selected variable domain. Nucleic acid sequences encoding the reconstituted antibody are introduced into a suitable cell line, such as a myeloma cell used for hybridoma production, such that human antibodies having the characteristics of monoclonal antibodies are secreted by the cell. Alternatively, monoclonal antibodies can be generated from mice that are transgenic for specific human heavy and light chain immunoglobulin genes. Such methods are known in the art. Most preferably the antibody is a humanized antibody. As used herein, a “humanized” antibody is one in which the complementarily-determining regions (CDR) of a mouse monoclonal antibody, which form the antigen binding loops of the antibody, are grafted onto the framework of a human antibody molecule. Owing to the similarity of the frameworks of mouse and human antibodies, it is generally accepted in the art that this approach produces a monoclonal antibody that is antigenically identical to a human antibody but binds the same antigen as the mouse monoclonal antibody from which the CDR sequences were derived. Methods for generating humanized antibodies are well known in the art. Humanized antibodies can also be generated using the antibody resurfacing technology. While the antibody employed in the conjugate of the inventive composition most preferably is a humanized monoclonal antibody, a human monoclonal antibody or a mouse monoclonal antibody, as described above, are also within the scope of the invention.

An antibody may be an antibody fragment. Antibody fragments that have at least one antigen binding site, and thus recognize and bind to at least one antigen or receptor present on the surface of a target cell, also are within the scope of the invention. In this respect, proteolytic cleavage of an intact antibody molecule can produce a variety of antibody fragments that retain the ability to recognize and bind antigens. For example, limited digestion of an antibody molecule with the protease papain typically produces three fragments, two of which are identical and are referred to as the Fab fragments, as they retain the antigen binding activity of the parent antibody molecule. Cleavage of an antibody molecule with the enzyme pepsin normally produces two antibody fragments, one of which retains both antigen-binding arms of the antibody molecule, and is thus referred to as the F(ab′)₂ fragment. A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. Antibody fragments of the present embodiments, however, are not limited to these exemplary types of antibody fragments. Any suitable antibody fragment that recognizes and binds to a desired cell surface receptor or antigen can be employed. Antibody-antigen binding can be assayed using any suitable method known in the art, such as, for example, radioimmunoassay (RIA), ELISA, Western blot, immunoprecipitation, and competitive inhibition assays.

In addition, the antibody can be a chimeric antibody. By “chimeric” is meant that the antibody comprises at least two immunoglobulins, or fragments thereof, obtained or derived from at least two different species (e.g., two different immunoglobulins, a human immunoglobulin constant region combined with a murine immunoglobulin variable region).

An “anti-tumor antibody” is an antibody that binds to a cancer or tumor antigen. The terms “cancer antigen” and “tumor antigen” are used interchangeably.

As used herein, the term “antigen” refers to a molecule capable of being bound by an antibody or a T cell receptor (TCR) if presented by MHC molecules. The term “antigen”, as used herein, also encompasses T-cell epitopes. At the molecular level, an antigen is characterized by its ability to be “bound” at the antigen-binding site of an antibody. An antigen is additionally capable of being recognized by the immune system. In some instances, an antigen is capable of inducing a humoral immune response. In some instances, an antigen is capable of inducing cellular immune response leading to the activation of B- and/or T-Iymphocytes. In some instance, an antigen may be antigenic and not immunogenic. For the purposes of the present embodiments, antigens are usually proteins or polysaccharides that includes parts (coats, capsules, cell walls, flagella, fimbrae, and toxins) of bacteria, viruses, and other microorganisms (e.g., a protein or polysaccharide derived from bacteria, virus, or other microorganism).

A “bacteriophage” is any one of a number of viruses that infect bacteria.

An “epitope”, also known as antigenic determinant, is the part of an antigen that is recognized by the immune system, specifically by antibodies, B cells, or T cells.

As used herein, “specific binding” refers to the property of the antibody, to: (1) to bind to the specific target antigen (e.g., a tumor antigen) with an affinity of at least 1×10⁷ M⁻¹, and (2) preferentially bind to the target antigen (e.g., a tumor antigen) with an affinity that is at least two-fold, 50-fold, 100-fold, 1000-fold, or more greater than its affinity for binding to a non-specific antigen (e.g., BSA, casein)

A “virus particle” (also known as virions) consist of two or three parts: the genetic material made from either DNA or RNA; a protein coat that protects these genes; and in some cases an envelope of lipids that surrounds the protein coat when they are outside a cell.

As used herein, the term “virus-like particle” refers to a structure resembling a virus particle. Moreover, a virus-like particle in accordance with the invention is non-replicative and noninfectious since it lacks all or part of the viral genome, in particular the replicative and infectious components of the viral genome. Virus-like particles refer to structures resembling a virus particle but which are not pathogenic. In general, virus-like particles lack the viral genome and, therefore, are noninfectious. Also, virus-like particles can be produced in large quantities by heterologous expression and can be easily purified.

A virus-like particle may contain nucleic acid distinct from their genome. A virus-like particle may be a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAg's have a spherical form of icosahedral symmetry. The term “capsid-like structure” as used herein, refers to a macromolecular assembly composed of viral protein subunits resembling the capsid morphology in the above defined sense but deviating from the typical symmetrical assembly while maintaining a sufficient degree of order and repetitiveness.

As used herein, the term “virus-like particle of a bacteriophage” refers to a virus-like par structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. Thus, for example, a reference to “a hostcell” includes a plurality of such host cells, and a reference to “an antibody” is a reference to one or more antibodies and equivalents thereof known to those skilled in the art, and so forth.

### REFERENCES

Aarnoudse C A, van den Doel P B, Heemskerk B, Schrier P I. Interleukin-2-induced, melanoma-specific T cells recognize CAMEL, an unexpected translation product of LAGE-1. Int J Cancer 1999; 82: 442-8. (PMID: 10399963)

Adair S J, Carr T M, Fink M J, Slingluff C L Jr, Hogan K T. The TAG family of cancer/testis antigens is widely expressed in a variety of malignancies and gives rise to HLA-A2-restricted epitopes. J Immunother 2008; 31: 7-17. (PMID: 18157007)

Ayyoub M, Stevanovic S, Sahin U, Guillaume P, Semis C, Rimoldi D, Valmori D, Romero P, Cerottini J C, Rammensee H G, Pfreundschuh M, Speiser D, Levy Proteasome-assisted identification of a SSX-2-derived epitope recognized by tumor-reactive CTL infiltrating metastatic melanoma. J Immunol 2002; 168: 1717-22. (PMID: 11823502)

Ayyoub M, Hesdorffer C S, Metthez G, Stevanovic S, Ritter G, Chen Y T, Old L J, Speiser D, Cerottini J C, Valmori D. Identification of an SSX-2 epitope presented by dendritic cells to circulating autologous CD4+ T cells. J Immunol 2004a; 172: 7206-11. (PMID: 15153546)

Ayyoub M, Hesdorffer C S, Montes M, Merlo A, Speiser D, Rimoldi D, Cerottini J C, Ritter G, Scanlan M, Old L J, Valmori D. An immunodominant SSX-2-derived epitope recognized by CD4+ T cells in association with HLA-DR. J Clin Invest 2004b; 113:1225-33. (PMID: 15085202)

Ayyoub M, Merlo A, Hesdorffer C S, Speiser D, Rimoldi D, Cerottini J C, Ritter G, Chen Y T, Old L J, Stevanovic S, Valmori D. Distinct but overlapping T helper epitopes in the 37-58 region of SSX-2. Clin Immunol 2005a; 114: 70-8. (PMID: 15596411)

Ayyoub M, Merle A, Hesdorffer C S, Rimoldi D, Speiser D, Cerottini J C, Chen Y T, Old L J, Stevanovic S, Valmori D. CD4+ T cell responses to SSX-4 in melanoma patients. J Immunol 2005b; 174: 5092-9. (PMID: 15814740)

Ayyoub M, Pignon P, Dojcinovic D, Raimbaud I, Old L J, Luescher I, Valmori D. Assessment of vaccine-induced CD4 T cell responses to the 119-143 immunodominant region of the tumor-specific antigen NY-ESO-1 using DRB1*0101 tetramers. Clin Cancer Res 2010; 16: 4607-15. (PMID: 20670945)

Benlalam H, Linard B, Guilloux Y, Moreau-Aubry A, Derré L, Diez E, Dreno B, Jotereau F, Labarrière N. Identification of five new HLA-B*3501-restricted epitopes derived from common melanoma-associated antigens, spontaneously recognized by tumor-infiltrating lymphocytes. J Immunol 2003; 171: 6283-9. (PMID: 14634146)

Bilsborough J, Panichelli C, Duffour M T, Warnier G, Lurquin C, Schultz E S, Thielemans K, Corthals J, Boon T, van der Bruggen P. A MAGE-3 peptide presented by HLA-B44 is also recognized by cytolytic T lymphocytes on HLA-B18. Tissue Antigens 2002; 60: 16-24. (PMID: 12366779)

Bioley G, Dousset C, Yeh A, Dupont B, Bhardwaj N, Mears G, Old L J, Ayyoub M, Valmori D. Vaccination with recombinant NY-ESO-1 protein elicits immunodominant HLA-DR52b-restricted CD4+ T cell responses with a conserved T cell receptor repertoire. Clin Cancer Res 2009; 15: 4467-74. (MID: 19531622)

Boel P, Wildmann C, Sensi M L, Brasseur R, Renauld J C, Coulie P, Boon T, van der Bruggen P. RAGE, a new gene encoding an antigen recognized on human melanomas by cytolytic lymphocytes. Immunity 1995; 2: 167-75. (PMID: 7895173)

Breckpot K, Heiman C. De Greef C, van der Bruggen P, Thielemans K. Identification of new antigenic peptide presented by HLA-Cw7 and encoded by several MAGE genes using dendritic cells transduced with lentiviruses. J Immunol 2004; 172: 2232-7. (PMID: 14764691)

Cesson V, Rivals J P, Escher A, Piotet E, Thielemans K, Posevitz V, Dojcinovic D, Monnier P, Speiser D, Bron L, Romero P. MAGE-A3 and MAGE-A4 specific CD4(+) T cells in head and neck cancer patients: detection of naturally acquired responses and identification of new epitopes. Cancer Immunol Immunother 2010; [Epub ahead of print]. (PMID: 20857101)

Chaux P, Luiten R, Demotte N, Vantomme V, Stroobant V, Traversari C, Russo V, Schultz E, Cornelis G R, Boon T, van der Bruggen P. Identification of five MAGE-A1 epitopes recognized by cytolytic T lymphocytes obtained by in vitro stimulation with dendritic cells transduced with MAGE-A1. J Immunol 1999a; 163: 2928-36. (PMID: 10453041)

Chaux P, Vantomme V, Stroobant V, Thielemans K, Corthals J, Luiten R, Eggermont A M, Boon T, van der Bruggen P. Identification of MAGE-3 epitopes presented by HLA-DR molecules to CD4(+) T lymphocytes. J Exp Med 1999b; 189: 767-78. (PMID: 10049940)

Chaux P, Lethé B, Van Snick J, Corthals J, Schultz E S, Cambiaso C L, Boon T, van der Bruggen P. A MAGE-1 peptide recognized on HLA-DR15 by CD4+ T cells. Eur J Immunol 2001; 31: 1910-6. (PMID: 11433388)

Chen J L, Dunbar P R, Gileadi. U, Jager E, Gnjatic S, Nagata Y, Stockert E, Panicali D L, Chen Y T, Knuth A, Old L J, Cerundolo V. Identification of NY-ESO-1 peptide analogues capable of improved stimulation of tumor-reactive CTL. J Immunol 2000; 165: 948-55. (PMID: 10878370)

Chen Q, Jackson H, Parente P, Luke T, Rizkalla. M, Tai T Y, Zhu H-C, Mifsud N A, Dimopoulos N, Masterman K-A, Hopkins W, Goldie H, Maraskovsky E, Green S, Miloradovic L, McCluskey J, Old L J, Davis I D, Cebon J, Chen W. Immunodominant CD4+ responses identified in a patient vaccinated with full-length NY-ESO-1 formulated with ISCOMATRIX adjuvant. Proc Natl Acad Sci USA 2004; 101: 9363-8. (PMID: 15197261)

Chiriva-Internati M, Wang Z, Pochopien S, Salati E, Lim S H. Identification of a sperm protein 17 CTL epitope restricted by HLA-A1. Int J Cancer 2003; 107: 863-5. (PMID: 14566839)

Consogno G, Manici S, Facchinetti V, Bachi A, Hammer J, Conti-Fine B M, Rugarli C, Traversari C, Protti M P. Identification of immunodominant regions among promiscuous HLA-DR-restricted CD4+ T-cell epitopes on the tumor antigen MAGE-3. Blood 2003; 101: 1038-44. (PMID: 12393675)

Corbière V, Nicolay H, Russo V, Stroobant V, Brichard V, Boon T, van der Bruggen P. Identification of a MAGE-1 peptide recognized by cytolytic T lymphocytes on HLA-B*5701 tumors. Tissue Antigens 2004; 63: 453-7. (PMID: 15104676)

De Backer O, Arden K C, Boretti M, Vantomme V, De Smet C, Czekay S, Viars C S, De Platin E, Brasseur F, Chomez P, Van den Eynde B, Boon T, van der Bruggen P. Characterization of the GAGE genes that are expressed in various human cancers and in normal testis. Cancer Res 1999; 59: 3157-65. (PMID: 10397259)

Duffour M T, Chaux P, Lurquin C, Cornelis G, Boon T, van der Bruggen P. A MAGE-A4 peptide presented by HLA-A2 is recognized by cytolytic T lymphocytes. Eur J Immunol 1999; 29: 3329-37. (PMID: 10540345)

Ebert L M, Liu Y C, Clements C S, Robson N C, Jackson H M, Markby J L, Dimopoulos N, Tan B S, Luescher I F, Davis I D, Rossjohn J, Cebon J, Purcell A W, Chen W. A long, naturally presented immunodominant epitope from NY-ESO-1 tumor antigen: implications for cancer vaccine design. Cancer Res 2009; 69: 1046-54. (PMID: 19176376)

Fukuyama T, Hanagiri T, Takenoyama M, Ichiki Y, Mizukami M, So T, Sugaya M, Sugio K, Yasumoto K. Identification of a new cancer/germline gene, KK-LC-1, encoding an antigen recognized by autologous CTL induced on human lung adenocarcinoma. Cancer Res 2006; 66: 4922-8. (PMID: 16651449)

Gaugler B, Van den Eynde B, van der Bruggen P, Romero P, Gaforio J J, De Plaen E, Lethe B, Brasseur F, Boon T. Human gene MAGE-3 codes for an antigen recognized on a melanoma by autologous cytolytic T lymphocytes. J Exp Med 1994; 179: 921-30. (PMID: 8113684)

Gnjatic S, Nagata Y, Jager E, Stockert E, Shankara S, Roberts B L, Mazzara G P, Lee S Y, Dunbar P R, Dupont B, Cerundolo V, Ritter G, Chen Y T, Knuth A, Old L J. Strategy for monitoring T cell responses to NY-ESO-1 in patients with any HLA class I allele. Proc Natl Acad Sci USA 2000; 97: 10917-22. (PMID: 11005863)

Godelaine D, Carrasco J, Brasseur F, Neyns B, Thielemans K, Boon T, Van Pel A. A new tumor-specific antigen encoded by MAGE-C2 and presented to cytolytic T lymphocytes by HLA-B44. Cancer Immunol Immunother 2007; 56: 753-9. (PMID: 17096150)

Guilloux Y, Lucas S, Brichard V G, Van Pel A, Viret C, De Plaen E, Brasseur F, Lethe B, Jotereau F, Boon T. A peptide recognized by human cytolytic T lymphocytes on HLA-A2 melanomas is encoded by an intron sequence of the N-acetylglucosaminyltransferase V gene. J Exp Med 1996; 183: 1173-83. (PMID: 8642259)

Hasegawa K, Noguchi Y, Koizumi F, Uenaka A, Tanaka M, Shimono M, Nakamura H, Shiku H, Gnjatic S, Murphy R, Hiramatsu Y, Old L J, Nakayama E. In vitro stimulation of CD8 and CD4 T cells by dendritic cells loaded with a complex of cholesterol-bearing hydrophobized pullulan and NY-ESO-1 protein: Identification of a new HLA-DR15-binding CD4 T-cell epitope. Clin Cancer Res 2006; 12: 1921-7. (PMID: 16551878)

Heidecker L, Brasseur F, Probst-Kepper M, Gueguen M, Boon T, Van den Eynde B J. Cytolytic T lymphocytes raised against a human bladder carcinoma recognize an antigen encoded by gene MAGE-A12. J Immunol 2000; 164: 6041-5. (PMID: 1.0820289)

Herman J, van der Bruggen P, Luescher I F, Mandruzzato S, Romero P, Thonnard J, Fleischhauer K, Boon T, Coolie P G. A peptide encoded by the human gene MAGE-3 and presented by HLA-B44 induces cytolytic T lymphocytes that recognize tumor cells expressing MAGE-3. Immunogenetics 1996; 43: 377-83. (PMID: 8606058)

Huang L Q, Brasseur F, Serrano A, De Plaen E, van der Bruggen P, Boon T, Van Pel A. Cytolytic T lymphocytes recognize an antigen encoded by MAGE-A10 on a human melanoma. J Immunol 1999; 162: 6849-54. (PMID: 10352307)

Janjic B, Andrade P, Wang X F, Fourcade J, Almunia C, Kudela P, Brufsky A, Jacobs S, Friedland D, Stoller R, Gillet D, Herberman R B, Kirkwood J M, Maillère B, Zarour H M. Spontaneous CD4+ T cell responses against TRAG-3 in patients with melanoma and breast cancers. J Immunol 2006; 177: 2717-27. (PMID: 16888034)

Jager F, Chen Y T, Drijfhout J W, Karbach J, Ringhoffer M, Jager D, Arand M, Wada H, Noguchi Y, Stockert E, Old L J, Knuth A. Simultaneous humoral and cellular immune response against cancer-testis antigen NY-ESO-1: definition of human histocompatibility leukocyte antigen (HLA)-A2-binding peptide epitopes. J Exp Med 1998; 187: 265-70. (PMID: 9432985)

Jager E, Jager D, Karbach J, Chen Y T, Ritter G, Nagata Y, Gnjatic S, Stockert E, Arand M, Old L J, Knuth A. Identification of NY-ESO-1 epitopes presented by human histocompatibility antigen (HLA)-DRB4*0101-0103 and recognized by CD4+ T lymphocytes of patients with NY-ESO-1-expressing melanoma. J Exp Med 2000; 191: 625-30. (PMID: 10684854)

Jager E, Karbach J, Gnjatic S, Jager D, Maeurer M, Atmaca A, Arand M, Skipper J, Stockert E, Chen Y T, Old L J, Knuth A. Identification of a naturally processed NY-ESO-1 peptide recognized by CD8+ T cells in the context of HLA-B51. Cancer Immun [serial online] 2002; 2: 12. URL: http://www.cancerimmunity.org/v2p12/020812.htm (PMID: 12747757)

Jerome K R, Domenech N, Finn O J. Tumor-specific cytotoxic T cell clones from patients with breast and pancreatic adenocarcinoma recognize EBV-immortalized B cells transfected with polymorphic epithelial mucin complementary DNA. J Immunol 1993; 151: 1654-62. (PMID: 8393050)

Kawashima I, Hudson S J, Tsai V, Southwood S, Takesako K, Appella E, Sette A, Celis E. The multi-epitope approach for immunotherapy for cancer: identification of several CTL epitopes from various tumor-associated antigens expressed on solid epithelial tumors. Hum Immunol 1998; 59: 1-14. (PMID: 9544234)

Knights A J, Nuber N, Thomson C W, de la Rosa O, Jager E, Tiercy J M, van den Broek M, Pascolo S, Knuth A, Zippelius A. Modified tumour antigen-encoding mRNA facilitates the analysis of naturally occurring and vaccine-induced CD4 and CD8 T cells in cancer patients. Cancer Immunol Immunother 2009; 58: 325-38. (PMID: 18663444)

Kobayashi H, Song Y, Hoon D S, Appella E, Celis E. Tumor-reactive T helper lymphocytes recognize a promiscuous MAGE-A3 epitope presented by various major histocompatibility complex class II alleles. Cancer Res 2001; 61: 4773-8. (MD: 11406551)

Kobayashi T, Lonchay C, Colau D, Demotte N, Boon T, van der Bruggen P. New MAGE-4 antigenic peptide recognized by cytolytic T lymphocytes on HLA-A1 tumor cells. Tissue Antigens 2003; 62: 426-32. (PMID: 14617050)

Luiten R, van der Bruggen P. A MAGE-A1 peptide is recognized on HLA-B7 human tumors by cytolytic T lymphocytes. Tissue Antigens 2000a; 55: 149-52. (PMID: 10746786)

Luiten R M, Demotte N, Tine J, van der Bruggen P. A MAGE-A1 peptide presented to cytolytic T lymphocytes by both HLA-B35 and HLA- A1 molecules. Tissue Antigens 2000b; 56: 77-81. (PMID: 10958359)

Lupetti R, Pisarra P, Verrecchia A, Farina C, Nicolini G, Anichini A, Bordignon C, Sensi M, Parmiani G, Traversari C. Translation of a retained intron in tyrosinase-related protein (TRP)-2 mRNA generates a new cytotoxic T lymphocyte (CTL)-defined and shared human melanoma antigen not expressed in normal cells of the melanocytic lineage. J Exp Med 1998; 188: 1005-16. (PMID: 9743519)

Ma W, Germeau C, Vigneron N, Maernoudt A S, Morel S, Boon T, Coulie P G, Van den Eynde B J. Two new tumor-specific antigenic peptides encoded by gene MAGE-C2 and presented to cytolytic T lymphocytes by HLA-A2. Int J Cancer 2004; 109: 698-702. (PMID: 14999777)

Mandic M, Castelli F, Janjic B, Almunia C, Andrade P, Gillet D, Brusic V, Kirkwood J M, Maillere B, Zarour H M. One NY-ESO-1-derived epitope that promiscuously binds to multiple HLA-DR and HLA-DP4 molecules and stimulates autologous CD4+ T cells from patients with NY-ESO-1-expressing melanoma. J Immunol 2005; 174: 1751-9. (PMID: 15661941)

Manici S, Sturniolo T, Imro M A, Hammer J, Sinigaglia F, Noppen C, Spagnoli G, Mazzi B, Bellone M, Dellabona P, Protti M P. Melanoma cells present a MAGE-3 epitope to CD4(+) cytotoxic T cells in association with histocompatibility leukocyte antigen DR11. J Exp Med 1999; 189: 871-6. (PMID: 10049951)

Matsuzaki J, Qian F, Luescher I, Lele S, Ritter G, Shrikant P A, Gnjatic S, Old L J, Odunsi K. Recognition of naturally processed and ovarian cancer reactive CD8+ T cell epitopes within a promiscuous HLA class II T-helper region of NY-ESO-1. Cancer Immunol Immunother 2008; 57: 1185-95. (PMID: 18253733)

Miyagawa N, Kono K, Mimura K, Omata H, Sugai H, Fujii H. A newly identified MAGE-3-derived, HLA-A24-restricted peptide is naturally processed and presented as a CTL epitope MAGE-3-expressing gastrointestinal cancer cells. Oncology 2006; 70: 54-62. (PMID: 16446550)

Miyahara Y, Naota H, Wang L, Hiasa A, Goto M, Watanabe M, Kitano S, Okumura S, Takemitsu T, Yuta A, Majima Y, Lemonnier F A, Boon T, Shiku H. Determination of cellularly processed HLA-A2402-restricted novel CTL epitopes derived from two cancer germ line genes, MAGE-A4 and SAGE. Clin Cancer Res 2005; 11: 5581-9. (W D: 16061876)

Mizote Y, Taniguchi T, Tanaka K, Isobe M, Wada H, Saika I, Kita S, Koide Y, Uenaka A, Nakayama E. Three novel NY-ESO-1 epitopes bound to DRB1*0803, DQB1*0401 and DRB1*0901 recognized by CD4 T cells from CHP-NY-ESO-1-vaccinated patients. Vaccine 2010; 28: 5338-46. (PMID: 20665979)

Monji M, Nakatsura T, Senju S, Yoshitake Y, Sawatsubashi M, Shinohara M, Kageshita T, Ono T, Inokuchi A, Nishimura Y. Identification of a novel human cancer/testis antigen, KM-HN-1, recognized by cellular and humoral immune responses. Clin Cancer Res 2004; 10: 6047-57. (PMID: 15447989)

Moreau-Aubry A, Le Guiner S, Labarriere N, Gesnel M C, Jotereau F, Breathnach R. A processed pseudogene codes for a new antigen recognized by a CD8(+) T cell clone on melanoma. J Exp Med 2000; 191: 1617-24. (PMID: 10790436)

Neumann F, Wagner C, Stevanovic S, Kubuschok B, Schormann C, Mischo A, Ertan K, Schmidt W, Pfreundschuh M. Identification of an HLA-DR-restricted peptide epitope with a promiscuous binding pattern derived from the cancer testis antigen HOM-MEL40/SSX2. Int J Cancer 2004; 112: 661-8. (PMID: 15382048)

Nuber N, Curioni-Fontecedro A, Matter C, Soldini D, Tiercy J M, von Boehmer L, Moch H, Dummer R, Knuth A, van den Broek M. Fine analysis of spontaneous MAGE-C1/CT7-specific immunity in melanoma patients. Proc Natl Acad Sci USA 2010; 107: 15187-92. (PMID: 20696919)

Oehlrich N, Devitt G, Linnebacher M, Schwitalle Y, Grosskinski S, Stevanovic S, Zoller M. Generation of RAGE-1 and MAGE-9 peptide-specific cytotoxic T-lymphocyte lines for transfer in patients with renal cell carcinoma. Int J Cancer 2005; 117: 256-64. (PMID: 15900605)

Oiso M, Eura M, Katsura F, Takiguchi M, Sobao Y, Masuyama K, Nakashima M, Itoh K, Ishikawa I. A newly identified MAGE-3-derived epitope recognized by HLA-A24-restricted cytotoxic T lymphocytes. Int J Cancer 1999; 81: 387-94. (PMID: 10209953)

Ottaviani S, Zhang Y, Boon T, van der Bruggen. A MAGE-1 antigenic peptide recognized by human cytolytic T lymphocytes on HLA-A2 tumor cells. Cancer Immunol Immunother 2005; 54: 1214-20. (PMID: 16025263)

Ottaviani S, Colau D, van der Bruggen P, der Bruggen P V. A new MAGE-4 antigenic peptide recognized by cytolytic T lymphocytes on HLA-A24 carcinoma cells. Cancer Immunol Immunother 2006; 55: 867-72. (PMID: 16151806)

Panelli M C, Bettinotti M P, Lally K, Ohnmacht G A, Li Y, Robbins P, Riker A, Rosenberg S A, Marincola F M. A tumor-infiltrating lymphocyte from a melanoma metastasis with decreased expression of melanoma differentiation antigens recognizes MAGE-12. J Immunol 2000; 164: 4382-92. (MD: 10754339)

Pascolo S, Schirle M, Guckel B, Dumrese T, Stumm S, Kayser S, Moris A, Wallwiener D, Rammensee H-G, Stevanovic S. A MAGE-A1 HLA-A A*0201 epitope identified by mass spectrometry. Cancer Res 2001;61: 4072-7. (PMID: 11.358828)

Rimoldi D, Rubio-Godoy V, Dutoit V, Lienard D, Salvi S, Guillaume P, Speiser D, Stockert E, Spagnoli G, Servis C, Cerottini J C, Lejeune F, Romero P, Valmori D. Efficient simultaneous presentation of NY-ESO-1/LAGE-1 primary and nonprimary open reading frame-derived CTL epitopes in melanoma. J Immunol 2000; 165: 7253-61. (PMID: 11120859)

Russo V, Tanzarella S, Dalerba P, Rigatti D, Rovere P, Villa A, Bordignon C, Traversari C. Dendritic cells acquire the MAGE-3 human tumor antigen from apoptotic cells and induce a class I-restricted T cell response. Proc Natl Acad Sci USA 2000; 97: 2185-90. (PMID: 10681453)

Schiavetti F, Thonnard J, Colau D, Boon T, Coulie P G. A human endogenous retroviral sequence encoding an antigen recognized on melanoma by cytolytic T lymphocytes. Cancer Research 2002; 62: 5510-6. (PMID: 12359761)

Schultz E S, Lethe B, Cambiaso C L, Van Snick J, Chaux P, Cordials J, Heiman C, Thielemans K, Boon T, van der Bruggen P. A MAGE-A3 peptide presented by HLA-DP4 is recognized on tumor cells by CD4+ cytolytic T lymphocytes. Cancer Res 2000; 60: 6272-5. (PMID: 11103782)

Schultz E S, Zhang Y, Knowles R, Tine J, Traversari C, Boon T, van der Bruggen P. A MAGE-3 peptide recognized on HLA-B35 and HLA-A1 by cytolytic T lymphocytes. Tissue Antigens 2001; 57: 103-9. (PMID: 11260504)

Schultz E S, Chapiro J, Lurquin C, Claverol S, Burlet-Schiltz O, Warnier G, Russo V, Morel S, Levy F, Boon T, Van den Eynde B J, van der Bruggen P. The production of a new MAGE-3 peptide presented to cytolytic T lymphocytes by HLA-B40 requires the immunoproteasome. J Exp Med 2002; 195: 391-9. (PMID: 11854353)

Schultz E S, Schuler-Thurner B, Stroobant V, Jenne L, Berger T G, Thielemans K, van der Bruggen P, Schuler G. Functional analysis of tumor-specific Th cell responses detected in melanoma patients after dendritic cell-based immunotherapy. J Immunol 2004; 172: 1304-10. (PMID: 14707109)

Shimono M, Uenaka A, Noguchi Y, Sato S, Okumura H, Nakagawa K, Kiura K, Tanimoto M, Nakayama E. Identification of DR9-restricted XAGE antigen on lung adenocarcinoma recognized by autologous CD4 T-cells. Int J Oncol 2007; 30: 835-40. (PMID: 17332921)

Slager E H, Borghi M, van der Minne C E, Aarnoudse C A, Havenga M J, Schrier P I, Osanto S, Griffioen M. CD4+ Th2 cell recognition of KLA-DR-restricted epitopes derived from CAMEL: a tumor antigen translated in an alternative open reading frame. J Immunol 2003; 170: 1490-7. (MID: 12538712)

Slager E H, van der Minne C E, Krüse M, Krueger D D, Griffioen M, Osanto S. Identification of multiple HLA-DR-restricted epitopes of the tumor-associated antigen CAMEL by CD4+ Th1/Th2 lymphocytes. J Immunol 2004a; 172: 5095-102. (PMID: 15067093)

Slager E H, van der Minne C E, Goudsmit J, van Oers J M M, Kostense S, Havenga M J E, Osanto S, Griffioen M. Induction of CAMEL/NY-ESO-ORF2-specific CD8+ T cells upon stimulation with dendritic cells infected with a modified Ad5 vector expressing a chimeric Ad5/35 fiber. Cancer Gene Ther 2004b; 11: 227-36. (PMID: 14726960)

Sun Z, Lethé B, Zhang Y, Russo V, Colau D, Stroobant V, Boon T, van der Bruggen P. A new LAGE-1 peptide recognized by cytolytic T lymphocytes on HLA-A68 tumors. Cancer Immunol Immunother 2006; 55: 644-52. (PMID: 16187088)

Tahara K, Takesako K, Sette A, Celis E, Kitano S, Akiyoshi T. Identification of a MAGE-2-encoded human leukocyte antigen-A24-binding synthetic peptide that induces specific antitumor cytotoxic T lymphocytes. Clin Cancer Res 1999; 5: 2236-41. (PMID: 10473111)

Tanzarella S, Russo V, Lionello I, Dalerba P, Rigatti D, Bordignon C, Traversari C. Identification of a promiscuous T cell epitope encoded by multiple members of the MAGE family. Cancer Res 1999; 59: 2668-74. (PMID: 10363990)

Traversari C, van der Bruggen P, Luescher I F, Lurquin C, Chomez P, Van Pel A, De Plaen E, Amar-Costesec A, Boon T. A nonapeptide encoded by human gene MAGE-1 is recognized on HLA-A1 by cytolytic T lymphocytes directed against tumor antigen MZ2-E. J Exp Med 1992; 176: 1453-7. (PMID; 1402688)

Valmori D, Dutoit V, Lienard D, Rimoldi D, Pittet M J, Champagne P, Ellefsen K, Sahin U, Speiser D, Lejeune F, Cerottini J C, Romero P. Naturally occurring human lymphocyte antigen-A2 restricted CD8+ T-cell response to the cancer testis antigen NY-ESO-1 in melanoma patients. Cancer Res 2000; 60: 4499-506. (PMID: 10969798)

Valmori D, Qian F, Ayyoub M, Renner C, Merin A, Gnjatic S, Stockert E, Driscoll D, Lele S, Old L J, Odunsi K. Expression of synovial sarcoma X (SSX) antigens in epithelial ovarian cancer and identification of SSX-4 epitopes recognized by CD4+ T cells. Clin Cancer Res 2006; 12: 398-404. (PMID: 16428478)

Van den Eynde B, Peeters O, De Backer O, Gaugler B, Lucas S, Boon T. A new family of genes coding for an antigen recognized by autologous cytolytic T lymphocytes on a human melanoma. J Exp Med 1995;182: 689-98. (PMID: 7544395)

van der Bruggen P, Szikora J P, Boel P, Wildmann C, Somville M, Sensi M, Boon T. Autologous cytolytic T lymphocytes recognize a MAGE-1 nonapeptide on melanomas expressing HLA-Cw*1601. Eur J Immunol 1994a.; 24: 2134-40. (PMID: 7522162)

van der Bruggen P, Bastin J, Gajewski T, Coulie P G, Boel P, De Smet C, Traversari C, Townsend A, Boon T. A peptide encoded by human gene MAGE-3 and presented by HLA-A2 induces cytolytic T lymphocytes that recognize tumor cells expressing MAGE-3. Eur J Immunol 1994b; 24: 3038-43. (PMID: 7805731)

Vantomme V, Boel P, De Plaen F, Boon T, van der Bruggen P. A new tumor-specific antigenic peptide encoded by MAGE-6 is presented to cytolytic T lymphocytes by HLA-Cw16. Cancer Immun [serial online] 2003; 3: 17. URL: http://www.cancerimmunity.org/v3p17/031118.htm (PMID: 14664500)

Wang H Y, Lee D A, Peng G, Guo Z, Li Y, Kiniwa Y, Shevach E M, Wang R F. Tumor-specific human CD4+ regulatory T cells and their ligands: implications for immunotherapy. Immunity 2004; 20: 107-18. (PMID: 14738769)

Wang R F, Johnston S L, Zeng G, Topalian S L, Schwartzentruber D J, Rosenberg S A. A breast and melanoma-shared tumor antigen: T cell responses to antigenic peptides translated from different open reading frames. J Immunol 1998; 161: 3598-606. (PMID: 9759882)

Wang X F, Cohen W M, Castelli F A, Almunia C, Lethé B, Pouvelle-Moratille S, Munier G, Charron D, Ménez A, Zarour H M, van der Bruggen P, Busson M, Maillère B. Selective identification of HLA-DP4 binding T cell epitopes encoded by the MAGE-A gene family. Cancer Immunol Immunother 2007; 56: 807-18. (PMID: 16988823)

Zarour H M, Storkus W J, Brusic V, Williams F, Kirkwood J M. NY-ESO-1 encodes DRB1*0401-restricted epitopes recognized by melanoma-reactive CD4+ T cells. Cancer Res 2000; 60: 4946-52. (MD: 10987311)

Zarour H M, Maillère B, Brusic V, Coval K, Williams E, Pouvelle-Moratille S, Castelli F, Land S, Bennouna J, Logan T, Kirkwood J M. NY-ESO-1 119-143 is a promiscuous major histocompatibility complex class II T-helper epitope recognized by Th1- and Th2-type tumor-reactive CD4+ T cells. Cancer Res 2002; 62: 213-8. (PMID: 11782380)

Zeng G, Touloukian C E, Wang X, Restifo N P, Rosenberg S A, Wang R F. Identification of CD4+ T cell epitopes from NY-ESO-1 presented by HLA-DR molecules. J Immunol 2000; 165: 1153-9. (PMID: 10878395)

Zeng G, Wang X, Robbins P F, Rosenberg S A, Wang R F. CD4(+) T cell recognition of MHC class II-restricted epitopes from NY-ESO-1 presented by a prevalent HLA DP4 allele: association with NY-ESO-1 antibody production. Proc Natl Acad Sci USA 2001; 98: 3964-9. (PMID: 11259659)

Zhang Y, Stroobant V, Russo V, Boon T, van der Bruggen P. A MAGE-A4 peptide presented by HLA-B37 is recognized on human tumors by cytolytic T lymphocytes. Tissue Antigens 2002; 60: 365-71. (PMID: 12492812)

Zhang Y, Chaux P, Stroobant V, Eggermont A M, Corthals J, Maillere B, Thielemans K, Marchand M, Boon T, Van Der Bruggen P. A MAGE-3 peptide presented by HLA-DR1 to CD4+ T cells that were isolated from a melanoma patient vaccinated with a MAGE-3 protein. J Immunol 2003; 171: 219-25. (PMID: 12817001)

Zorn E, Hercend T. A MAGE-6-encoded peptide is recognized by expanded lymphocytes infiltrating a spontaneously regressing, human primary melanoma lesion. Eur J Immunol 1999; 29: 602-7. (PMID: 10064076)

What is claimed is:

1. An immunoconjugates comprising an antibody and one or more immune enhancers, wherein the antibody is specific for a tumor antigen, and wherein the immune enhancer is an antigen derived from a viral entity or bacteria.

2. The immunoconjugates of claim 1, wherein the viral entity is a non-infectious, non-replicating virus, viral particle, virus-like particle (VLP), or antigenic component thereof.

3. The immunoconjugates of claim 1, wherein the immune enhancer is a bacteriophage, bacteriophage particle, bacteriophage VLP.

4. The immunoconjugates of claim 1, wherein the immune enhancer is a lambda phage or lambda phage particle.

5. The immunoconjugates of claim 1, wherein the immune enhancer is a mycobacterial antigen.

6. The immunoconjugates of claim 1, wherein the immune enhancer is a tuberculosis (TB) antigen.

7. The immunoconjugates of claim 1, wherein the tumor antigen is aspartyl (asparaginyl) beta-hydroxylase (HAAH) polypeptide.

8. The immunoconjugates of claim 1, wherein the immune enhancer is capable of promoting the recruitment of immune system components and systemic immune response.

9. A method of treating cancer comprising providing a patient with an immunoconjugates comprising an antibody and one or more immune enhancers, wherein the antibody is specific for a tumor antigen, and wherein the immune enhancer is an antigen derived from a viral entity or bacteria.

10. The method of claim 9, wherein the viral entity is a non-infectious, non-replicating virus, viral particle, virus-like particle (VLP), or antigenic component thereof.

11. The method of claim 9, wherein the immune enhancer is a bacteriophage, bacteriophage particle, bacteriophage VLP.

12. The method of claim 9, wherein the immune enhancer is a lambda phage or lambda phage particle.

13. The method of claim 9, wherein the immune enhancer is a mycobacterial antigen.

14. The method of claim 9, wherein the immune enhancer is a bacterial antigen.

15. The method of claim 9, wherein the immune enhancer is a fungal antigen.

16. The method of claim 9, wherein the immune enhancer is a parasite antigen.

17. The method of claim 9, wherein the immune enhancer is a tuberculosis (TB) antigen.

18. The method of claim 9, wherein the tumor antigen is aspartyl (asparaginyl) beta-hydroxylase (HAAH) polypeptide.

19. The method of claim 9, wherein the immune enhancer is capable of promoting the recruitment of immune system components and systemic immune response.

20. An immunoconjugate comprising an antibody to the tumor antigen aspartyl (asparaginyl) beta-hydroxylase (HAAH) polypeptide conjugated to both a non-replicating Lambda virus and at least one TB antigen.

21. The immunoconjugates of claim 20, wherein the TB antigen is selected from one or more of the following: ESAT-6, Ag85A, AG85B, MPT51, MPT64, CFP10, TB10.4, Mtb8.4, hspX, CFP6, Mtb12, Mtb9.9 antigens, Mtb32A, PstS-1, PstS-2, PstS-3, MPT63, Mtb39, Mtb41, MPT83, 71-kDa, PPE 68, LppX, and antigenic portions thereof.


SEQUENCE LISTING (142 sequences)

SEQ ID NO: 1 - protein, 9 aa, Unknown
  Description of Unknown Tumor-specific antigen
AARAVFLAL

SEQ ID NO: 2 - protein, 8 aa, Unknown
  Description of Unknown Tumor-specific antigen
YRPRPRRY

SEQ ID NO: 3 - protein, 9 aa, Unknown
  Description of Unknown Tumor-specific antigen
YYWPRPRRY

SEQ ID NO: 4 - protein, 10 aa, Unknown
  Description of Unknown Tumor-specific antigen
VLPDVFIRCV

SEQ ID NO: 5 - protein, 9 aa, Unknown
  Description of Unknown Tumor-specific antigen
MLAVISCAV

SEQ ID NO: 6 - protein, 9 aa, Unknown
  Description of Unknown Tumor-specific antigen
RQKRILVNL

SEQ ID NO: 7 - protein, 9 aa, Unknown
  Description of Unknown Tumor-specific antigen
NYNNFYRFL

SEQ ID NO: 8 - protein, 10 aa, Unknown
  Description of Unknown Tumor-specific antigen
EYSKECLKEF

SEQ ID NO: 9 - protein, 9 aa, Unknown
  Description of Unknown Tumor-specific antigen
EYLSLSDKI

SEQ ID NO: 10 - protein, 11 aa, Unknown
  Description of Unknown Tumor-specific antigen
MLMAQEALAFL

SEQ ID NO: 11 - protein, 9 aa, Unknown
  Description of Unknown Tumor-specific antigen
SLLMWITQC

SEQ ID NO: 12 - protein, 10 aa, Unknown
  Description of Unknown Tumor-specific antigen
LAAQERRVPR

SEQ ID NO: 13 - protein, 9 aa, Unknown
  Description of Unknown Tumor-specific antigen
ELVRRILSR

SEQ ID NO: 14 - protein, 9 aa, Unknown
  Description of Unknown Tumor-specific antigen
APRGVRMAV

SEQ ID NO: 15 - protein, 14 aa, Unknown
  Description of Unknown Tumor-specific antigen
SLLMWITQCFLPVF

SEQ ID NO: 16 - protein, 20 aa, Unknown
  Description of Unknown Tumor-specific antigen
QGAMLAAQERRVPRAAEVPR

SEQ ID NO: 17 - protein, 18 aa, Unknown
  Description of Unknown Tumor-specific antigen
AADHRQLQLSISSCLQQL

SEQ ID NO: 18 - protein, 22 aa, Unknown
  Description of Unknown Tumor-specific antigen
CLSRRPWKRSWSAGSCPGMPHL

SEQ ID NO: 19 - protein, 22 aa, Unknown
  Description of Unknown Tumor-specific antigen
CLSRRPWKRSWSAGSCPGMPHL

SEQ ID NO: 20 - protein, 13 aa, Unknown
  Description of Unknown Tumor-specific antigen
ILSRDAAPLPRPG

SEQ ID NO: 21 - protein, 14 aa, Unknown
  Description of Unknown Tumor-specific antigen
AGATGGRGPRGAGA

SEQ ID NO: 22 - protein, 9 aa, Unknown
  Description of Unknown Tumor-specific antigen
EADPTGHSY

SEQ ID NO: 23 - protein, 9 aa, Unknown
  Description of Unknown Tumor-specific antigen
KVLEYVIKV

SEQ ID NO: 24 - protein, 9 aa, Unknown
  Description of Unknown Tumor-specific antigen
SLFRAVITK

SEQ ID NO: 25 - protein, 10 aa, Unknown
  Description of Unknown Tumor-specific antigen
EVYDGREHSA

SEQ ID NO: 26 - protein, 9 aa, Unknown
  Description of Unknown Tumor-specific antigen
RVRFFFPSL

SEQ ID NO: 27 - protein, 9 aa, Unknown
  Description of Unknown Tumor-specific antigen
EADPTGHSY

SEQ ID NO: 28 - protein, 10 aa, Unknown
  Description of Unknown Tumor-specific antigen
REPVTKAEML

SEQ ID NO: 29 - protein, 9 aa, Unknown
  Description of Unknown Tumor-specific antigen
DPARYEFLW

SEQ ID NO: 30 - protein, 11 aa, Unknown
  Description of Unknown Tumor-specific antigen
ITKKVADLVGF

SEQ ID NO: 31 - protein, 9 aa, Unknown
  Description of Unknown Tumor-specific antigen
SAFPTTINF

SEQ ID NO: 32 - protein, 9 aa, Unknown
  Description of Unknown Tumor-specific antigen
SAYGEPRKL

SEQ ID NO: 33 - protein, 9 aa, Unknown
  Description of Unknown Tumor-specific antigen
SAYGEPRKL

SEQ ID NO: 34 - protein, 15 aa, Unknown
  Description of Unknown Tumor-specific antigen
TSCILESLFRAVITK

SEQ ID NO: 35 - protein, 15 aa, Unknown
  Description of Unknown Tumor-specific antigen
PRALAETSYVKVLEY

SEQ ID NO: 36 - protein, 16 aa, Unknown
  Description of Unknown Tumor-specific antigen
FLLLKYRAREPVTKAE

SEQ ID NO: 37 - protein, 12 aa, Unknown
  Description of Unknown Tumor-specific antigen
EYVIKVSARVRF

SEQ ID NO: 38 - protein, 10 aa, Unknown
  Description of Unknown Tumor-specific antigen
YLQLVFGIEV

SEQ ID NO: 39 - protein, 9 aa, Unknown
  Description of Unknown Tumor-specific antigen
EYLQLVFGI

SEQ ID NO: 40 - protein, 10 aa, Unknown
  Description of Unknown Tumor-specific antigen
REPVTKAEML

SEQ ID NO: 41 - protein, 9 aa, Unknown
  Description of Unknown Tumor-specific antigen
EGDCAPEEK

SEQ ID NO: 42 - protein, 14 aa, Unknown
  Description of Unknown Tumor-specific antigen
LLKYRAREPVTKAE

SEQ ID NO: 43 - protein, 9 aa, Unknown
  Description of Unknown Tumor-specific antigen
EVDPIGHLY

SEQ ID NO: 44 - protein, 9 aa, Unknown
  Description of Unknown Tumor-specific antigen
FLWGPRALV

SEQ ID NO: 45 - protein, 9 aa, Unknown
  Description of Unknown Tumor-specific antigen
KVAELVHFL

SEQ ID NO: 46 - protein, 9 aa, Unknown
  Description of Unknown Tumor-specific antigen
TFPDLESEF

SEQ ID NO: 47 - protein, 9 aa, Unknown
  Description of Unknown Tumor-specific antigen
VAELVHFLL

SEQ ID NO: 48 - protein, 10 aa, Unknown
  Description of Unknown Tumor-specific antigen
MEVDPIGHLY

SEQ ID NO: 49 - protein, 9 aa, Unknown
  Description of Unknown Tumor-specific antigen
EVDPIGHLY

SEQ ID NO: 50 - protein, 10 aa, Unknown
  Description of Unknown Tumor-specific antigen
REPVTKAEML

SEQ ID NO: 51 - protein, 9 aa, Unknown
  Description of Unknown Tumor-specific antigen
AELVHFLLL

SEQ ID NO: 52 - protein, 10 aa, Unknown
  Description of Unknown Tumor-specific antigen
MEVDPIGHLY

SEQ ID NO: 53 - protein, 9 aa, Unknown
  Description of Unknown Tumor-specific antigen
WQYFFPVIF

SEQ ID NO: 54 - protein, 9 aa, Unknown
  Description of Unknown Tumor-specific antigen
EGDCAPEEK

SEQ ID NO: 55 - protein, 16 aa, Unknown
  Description of Unknown Tumor-specific antigen
KKLLTQHFVQENYLEY

SEQ ID NO: 56 - protein, 16 aa, Unknown
  Description of Unknown Tumor-specific antigen
KKLLTQHFVQENYLEY

SEQ ID NO: 57 - protein, 16 aa, Unknown
  Description of Unknown Tumor-specific antigen
ACYEFLWGPRALVETS

SEQ ID NO: 58 - protein, 15 aa, Unknown
  Description of Unknown Tumor-specific antigen
RKVAELVHFLLLKYR

SEQ ID NO: 59 - protein, 12 aa, Unknown
  Description of Unknown Tumor-specific antigen
VIFSKASSSLQL

SEQ ID NO: 60 - protein, 12 aa, Unknown
  Description of Unknown Tumor-specific antigen
VIFSKASSSLQL

SEQ ID NO: 61 - protein, 15 aa, Unknown
  Description of Unknown Tumor-specific antigen
VFGIELMEVDPIGHL

SEQ ID NO: 62 - protein, 15 aa, Unknown
  Description of Unknown Tumor-specific antigen
GDNQIMPKAGLLIIV

SEQ ID NO: 63 - protein, 15 aa, Unknown
  Description of Unknown Tumor-specific antigen
TSYVKVLHHMVKISG

SEQ ID NO: 64 - protein, 16 aa, Unknown
  Description of Unknown Tumor-specific antigen
RKVAELVHFLLLKYRA

SEQ ID NO: 65 - protein, 16 aa, Unknown
  Description of Unknown Tumor-specific antigen
FLLLKYRAREPVTKAE

SEQ ID NO: 66 - protein, 9 aa, Unknown
  Description of Unknown Tumor-specific antigen
EVDPASNTY

SEQ ID NO: 67 - protein, 10 aa, Unknown
  Description of Unknown Tumor-specific antigen
GVYDGREHTV

SEQ ID NO: 68 - protein, 9 aa, Unknown
  Description of Unknown Tumor-specific antigen
NYKRCFPVI

SEQ ID NO: 69 - protein, 8 aa, Unknown
  Description of Unknown Tumor-specific antigen
SESLKMIF

SEQ ID NO: 70 - protein, 9 aa, Unknown
  Description of Unknown Tumor-specific antigen
MVKISGGPR

SEQ ID NO: 71 - protein, 9 aa, Unknown
  Description of Unknown Tumor-specific antigen
EVDPIGHVY

SEQ ID NO: 72 - protein, 10 aa, Unknown
  Description of Unknown Tumor-specific antigen
REPVTKAEML

SEQ ID NO: 73 - protein, 9 aa, Unknown
  Description of Unknown Tumor-specific antigen
EGDCAPEEK

SEQ ID NO: 74 - protein, 9 aa, Unknown
  Description of Unknown Tumor-specific antigen
ISGGPRISY

SEQ ID NO: 75 - protein, 14 aa, Unknown
  Description of Unknown Tumor-specific antigen
LLKYRAREPVTKAE

SEQ ID NO: 76 - protein, 9 aa, Unknown
  Description of Unknown Tumor-specific antigen
ALSVMGVYV

SEQ ID NO: 77 - protein, 9 aa, Unknown
  Description of Unknown Tumor-specific antigen
GLYDGMEHL

SEQ ID NO: 78 - protein, 9 aa, Unknown
  Description of Unknown Tumor-specific antigen
DPARYEFLW

SEQ ID NO: 79 - protein, 9 aa, Unknown
  Description of Unknown Tumor-specific antigen
FLWGPRALV

SEQ ID NO: 80 - protein, 9 aa, Unknown
  Description of Unknown Tumor-specific antigen
VRIGHLYIL

SEQ ID NO: 81 - protein, 9 aa, Unknown
  Description of Unknown Tumor-specific antigen
EGDCAPEEK

SEQ ID NO: 82 - protein, 15 aa, Unknown
  Description of Unknown Tumor-specific antigen
REPFTKAEMLGSVIR

SEQ ID NO: 83 - protein, 14 aa, Unknown
  Description of Unknown Tumor-specific antigen
AELVHFLLLKYRAR

SEQ ID NO: 84 - protein, 13 aa, Unknown
  Description of Unknown Tumor-specific antigen
SSALLSIFQSSPE

SEQ ID NO: 85 - protein, 9 aa, Unknown
  Description of Unknown Tumor-specific antigen
SFSYTLLSL

SEQ ID NO: 86 - protein, 9 aa, Unknown
  Description of Unknown Tumor-specific antigen
VSSFFSYTL

SEQ ID NO: 87 - protein, 10 aa, Unknown
  Description of Unknown Tumor-specific antigen
LLFGLALIEV

SEQ ID NO: 88 - protein, 9 aa, Unknown
  Description of Unknown Tumor-specific antigen
ALKDVEERV

SEQ ID NO: 89 - protein, 9 aa, Unknown
  Description of Unknown Tumor-specific antigen
SESIKKKVL

SEQ ID NO: 90 - protein, 20 aa, Unknown
  Description of Unknown Tumor-specific antigen
PDTRPAPGSTAPPAHGVTSA

SEQ ID NO: 91 - protein, 9 aa, Unknown
  Description of Unknown Tumor-specific antigen
QGQHFLQKV

SEQ ID NO: 92 - protein, 9 aa, Unknown
  Description of Unknown Tumor-specific antigen
SLLMWITQC

SEQ ID NO: 93 - protein, 11 aa, Unknown
  Description of Unknown Tumor-specific antigen
MLMAQEALAFL

SEQ ID NO: 94 - protein, 10 aa, Unknown
  Description of Unknown Tumor-specific antigen
ASGPGGGAPR

SEQ ID NO: 95 - protein, 10 aa, Unknown
  Description of Unknown Tumor-specific antigen
LAAQERRVPR

SEQ ID NO: 96 - protein, 10 aa, Unknown
  Description of Unknown Tumor-specific antigen
TVSGNILTIR

SEQ ID NO: 97 - protein, 13 aa, Unknown
  Description of Unknown Tumor-specific antigen
APRGPHGGAASGL

SEQ ID NO: 98 - protein, 9 aa, Unknown
  Description of Unknown Tumor-specific antigen
MPFATPMEA

SEQ ID NO: 99 - protein, 12 aa, Unknown
  Description of Unknown Tumor-specific antigen
KEFTVSGNILTI

SEQ ID NO: 100 - protein, 9 aa, Unknown
  Description of Unknown Tumor-specific antigen
MPFATPMEA

SEQ ID NO: 101 - protein, 9 aa, Unknown
  Description of Unknown Tumor-specific antigen
LAMPFATPM

SEQ ID NO: 102 - protein, 9 aa, Unknown
  Description of Unknown Tumor-specific antigen
ARGPESRLL

SEQ ID NO: 103 - protein, 14 aa, Unknown
  Description of Unknown Tumor-specific antigen
SLLMWITQCFLPVF

SEQ ID NO: 104 - protein, 25 aa, Unknown
  Description of Unknown Tumor-specific antigen
LLEFYLAMPFATPMEAELARRSLAQ

SEQ ID NO: 105 - protein, 25 aa, Unknown
  Description of Unknown Tumor-specific antigen
LLEFYLAMPFATPMEAELARRSLAQ

SEQ ID NO: 106 - protein, 12 aa, Unknown
  Description of Unknown Tumor-specific antigen
EFYLAMPFATPM

SEQ ID NO: 107 - protein, 25 aa, Unknown
  Description of Unknown Tumor-specific antigen
PGVLLKEFTVSGNILTIRLTAADHR

SEQ ID NO: 108 - protein, 12 aa, Unknown
  Description of Unknown Tumor-specific antigen
RLLEFYLAMPFA

SEQ ID NO: 109 - protein, 20 aa, Unknown
  Description of Unknown Tumor-specific antigen
QGAMLAAQERRVPRAAEVPR

SEQ ID NO: 110 - protein, 13 aa, Unknown
  Description of Unknown Tumor-specific antigen
PFATPMEAELARR

SEQ ID NO: 111 - protein, 20 aa, Unknown
  Description of Unknown Tumor-specific antigen
PGVLLKEFTVSGNILTIRLT

SEQ ID NO: 112 - protein, 10 aa, Unknown
  Description of Unknown Tumor-specific antigen
VLLKEFTVSG

SEQ ID NO: 113 - protein, 18 aa, Unknown
  Description of Unknown Tumor-specific antigen
AADHRQLQLSISSCLQQL

SEQ ID NO: 114 - protein, 25 aa, Unknown
  Description of Unknown Tumor-specific antigen
LLEFYLAMPFATPMEAELARRSLAQ

SEQ ID NO: 115 - protein, 15 aa, Unknown
  Description of Unknown Tumor-specific antigen
LKEFTVSGNILTIRL

SEQ ID NO: 116 - protein, 25 aa, Unknown
  Description of Unknown Tumor-specific antigen
PGVLLKEFTVSGNILTIRLTAADHR

SEQ ID NO: 117 - protein, 25 aa, Unknown
  Description of Unknown Tumor-specific antigen
LLEFYLAMPFATPMEAELARRSLAQ

SEQ ID NO: 118 - protein, 11 aa, Unknown
  Description of Unknown Tumor-specific antigen
KEFTVSGNILT

SEQ ID NO: 119 - protein, 14 aa, Unknown
  Description of Unknown Tumor-specific antigen
LLEFYLAMPFATPM

SEQ ID NO: 120 - protein, 14 aa, Unknown
  Description of Unknown Tumor-specific antigen
AGATGGRGPRGAGA

SEQ ID NO: 121 - protein, 9 aa, Unknown
  Description of Unknown Tumor-specific antigen
LYATVIHDI

SEQ ID NO: 122 - protein, 9 aa, Unknown
  Description of Unknown Tumor-specific antigen
ILDSSEEDK

SEQ ID NO: 123 - protein, 9 aa, Unknown
  Description of Unknown Tumor-specific antigen
KASEKIFYV

SEQ ID NO: 124 - protein, 16 aa, Unknown
  Description of Unknown Tumor-specific antigen
EKIQKAFDDIAKYFSK

SEQ ID NO: 125 - protein, 18 aa, Unknown
  Description of Unknown Tumor-specific antigen
WEKMKASEKIFYVYMKRK

SEQ ID NO: 126 - protein, 15 aa, Unknown
  Description of Unknown Tumor-specific antigen
KIFYVYMKRKYEAMT

SEQ ID NO: 127 - protein, 14 aa, Unknown
  Description of Unknown Tumor-specific antigen
KIFYVYMKRKYEAM

SEQ ID NO: 128 - protein, 20 aa, Unknown
  Description of Unknown Tumor-specific antigen
INKTSGPKRGKHAWTHRLRE

SEQ ID NO: 129 - protein, 20 aa, Unknown
  Description of Unknown Tumor-specific antigen
YFSKKEWEKMKSSEKIVYVY

SEQ ID NO: 130 - protein, 20 aa, Unknown
  Description of Unknown Tumor-specific antigen
MKLNYEVMTKLGFKVTLPPF

SEQ ID NO: 131 - protein, 20 aa, Unknown
  Description of Unknown Tumor-specific antigen
KHAWTHRLRERKQLVVYEEI

SEQ ID NO: 132 - protein, 20 aa, Unknown
  Description of Unknown Tumor-specific antigen
LGFKVTLPPFMRSKRAADFH

SEQ ID NO: 133 - protein, 20 aa, Unknown
  Description of Unknown Tumor-specific antigen
KSSEKIVYVYMKLNYEVMTK

SEQ ID NO: 134 - protein, 20 aa, Unknown
  Description of Unknown Tumor-specific antigen
KHAWTHRLRERKQLVVYEEI

SEQ ID NO: 135 - protein, 9 aa, Unknown
  Description of Unknown Tumor-specific antigen
SLGWLFLLL

SEQ ID NO: 136 - protein, 9 aa, Unknown
  Description of Unknown Tumor-specific antigen
LSRLSNRLL

SEQ ID NO: 137 - protein, 9 aa, Unknown
  Description of Unknown Tumor-specific antigen
LSRLSNRLL

SEQ ID NO: 138 - protein, 15 aa, Unknown
  Description of Unknown Tumor-specific antigen
CEFHACWPAFTVLGE

SEQ ID NO: 139 - protein, 15 aa, Unknown
  Description of Unknown Tumor-specific antigen
CEFHACWPAFTVLGE

SEQ ID NO: 140 - protein, 15 aa, Unknown
  Description of Unknown Tumor-specific antigen
CEFHACWPAFTVLGE

SEQ ID NO: 141 - protein, 10 aa, Unknown
  Description of Unknown Tumor-specific antigen
EVISCKLIKR

SEQ ID NO: 142 - protein, 17 aa, Unknown
  Description of Unknown Tumor-specific antigen
CATWKVICKSCISQTPG